SEQUENCE LISTING (12 sequences)

SEQ ID NO: 1 - DNA, 22 bp, synthetic construct
  source 1..22: mol_type = other DNA
tatttcgcccactaagccaatc

SEQ ID NO: 2 - DNA, 24 bp, synthetic construct
  source 1..24: mol_type = other DNA
cggatgctacttgtccaatgatgg

SEQ ID NO: 3 - DNA, 25 bp, synthetic construct
  source 1..25: mol_type = other DNA
gcaatctgttagtaggcagtggtgg

SEQ ID NO: 4 - DNA, 23 bp, synthetic construct
  source 1..23: mol_type = other DNA
gcaggaactgaaagaagtgggtg

SEQ ID NO: 5 - DNA, 20 bp, synthetic construct
  source 1..20: mol_type = other DNA
cacatgaagcagcacgactt

SEQ ID NO: 6 - DNA, 20 bp, synthetic construct
  source 1..20: mol_type = other DNA
tccttgaagtcgatgccctt

SEQ ID NO: 7 - DNA, 20 bp, synthetic construct
  source 1..20: mol_type = other DNA
gaacgaaaacgctggttagc

SEQ ID NO: 8 - DNA, 20 bp, synthetic construct
  source 1..20: mol_type = other DNA
cccggcaaaacaggtagtta

SEQ ID NO: 9 - DNA, 20 bp, synthetic construct
  source 1..20: mol_type = other DNA
cgtttccgttcttcttcgtc

SEQ ID NO: 10 - DNA, 20 bp, synthetic construct
  source 1..20: mol_type = other DNA
tgttcattccacggacaaaa

SEQ ID NO: 11 - DNA, 20 bp, synthetic construct
  source 1..20: mol_type = other DNA
caactcaatcgacagctgga

SEQ ID NO: 12 - DNA, 20 bp, synthetic construct
  source 1..20: mol_type = other DNA
ggctttgtttgccgtaatgt

(19) United States
(12) Patent Application Publication
(10) Pub. No.: US 2024/0425910 A1
(43) Pub. Date: Dec. 26, 2024

(54) KITS AND METHODS FOR QUANTITATIVE ASSESSMENT AND ENRICHMENT OF EXTRACHROMOSOMAL CIRCULAR DNA

(71) Applicant: CEDARS-SINAI MEDICAL CENTER, Los Angeles, CA (US)

(72) Inventors: Hisashi Tanaka, Los Angeles, CA (US); Lila Montoya, Los Angeles, CA (US); Armando E. Giuliano, Los Angeles, CA (US)

(73) Assignee: CEDARS-SINAI MEDICAL CENTER, Los Angeles, CA (US)

(21) Appl. No.: 18/706,126

(22) PCT Filed: Nov. 3, 2022
(86) PCT No.: PCT/US2022/079245

§ 371 Date: Apr. 30, 2024

Related U.S. Application Data
(60) Provisional application No. 63/276,346, filed on Nov. 5, 2021.

(51) Int. Cl. C12Q 1/6851; C12N 9/22; C12Q 1/6806

(52) U.S. Cl. CPC C12Q 1/6851; C12N 9/22; C12Q 1/6806; C12Y 301/11005; C12Y 301/27005; C12Y 304/21064

(57) ABSTRACT

Methods and reagents for related kits are provided for assessing enrichment of extrachromosomal circular DNA (ec-cDNA) isolated from biological samples. The methods include spiking the biological samples with a known proportion of linearized and circular plasmid DNA molecules and measuring their relative amounts post-exonuclease treatment as a benchmark to validate enrichment of eccDNA in the isolation process. Alternatively, or in combination, the method includes measuring the relative amounts of endogenous mitochondrial DNA and chromosomal DNA post-exonuclease treatment to assess the enrichment of eccDNA in the isolation process.

### CROSS-REFERENCE TO RELATED APPLICATIONS

This application includes a claim of priority under 35 U.S.C. § 119(e) to U.S. provisional patent application No. 63/276,346, filed Nov. 5, 2021, the entirety of which is hereby incorporated by reference.

### STATEMENT REGARDING FEDERALLY SPONSORED RESEARCH OR DEVELOPMENT

This invention was made with government support under Grant Nos. CA149385 and CA188111 awarded by National Institutes of Health. The Government has certain rights in the invention.

### REFERENCE TO SEQUENCE LISTING

This application contains a Sequence Listing submitted as an electronic file named “065472_000870WOPT_Sequence_Listing_ST26.xml”, having a size in bytes of 11,568 bytes, and created on Nov. 3, 2022. The information contained in this electronic file is hereby incorporated by reference in its entirety.

### FIELD OF INVENTION

This invention relates to isolation, enrichment, and assessment of extrachromosomal circular DNA at the naïve state.

### BACKGROUND

In eukaryotes, the vast majority of genetic information is encoded in the linear chromosomes of the nucleus, where circular DNA elements, or extrachromosomal circular DNA (eccDNA), are also present. eccDNA could modulate the genetic information of linear chromosomes and, as a result, phenotypes. This is the case with the most extensively studied extrachromosomal circular chromosomes, double minutes (DMs), which are several megabases (Mb) in size and are heavily implicated in cancer progression. DMs carry amplified oncogenes and DNA regulatory elements such as enhancers that promote the therapeutic resistance and progression of tumors. This phenotypic plasticity is associated with the remodeling of cancer genomes, as DMs can emerge from linear chromosomes, circularize, and reintegrate back into the genome. In contrast, the biological significance of small-sized eccDNA populations found in both cancer and non-cancer genomes remains unclear, despite the recent surge of nucleotide-level data from next-generation sequencing (NGS)-based studies.

Uncovering the expanding biological roles of eccDNA necessitates a delineation of their characteristics, including their chromosomal compositions and prevalence in cells. A barrier to fully understanding eccDNA biology could be associated with eccDNA enrichment approaches that rely on multiple displacement amplification (MDA), a non-PCR technique that amplifies DNA with a highly processive Φ29 DNA polymerase. When the template DNA is circular, DNA synthesis continues indefinitely, which is known as rolling-circle amplification (RCA). While RCA amplifies circular DNA, it often over-represents small-sized populations of eccDNA, rendering the analysis of in vitro amplified DNA qualitative, and not quantitative. RCA also eliminates epigenetic information associated with template DNA, leaving crucial biological information unaddressed. Furthermore, calling eccDNA is based on identifying sequencing reads that span the fusion points of circular DNA, which requires that discordant reads are confidently mapped to single-copy genomic regions.

Therefore, it is an objective of the present invention to provide for an isolation or enrichment method that circumvents the issues above and allows for a quantitative assessment of native eccDNA.

It is another objective of the present invention to provide for a system or a kit for the enrichment and assessment of native eccDNA.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for an assessment kit for use with a sample containing circular DNA, comprising: a. a first plasmid DNA molecule which is linearized, or a first plasmid DNA molecule and one or more restriction endonucleases to linearize the first plasmid DNA; and one or more of: b. a second plasmid DNA molecule having a size between 1,000 bp and 9,999 bp; c. a third plasmid DNA molecule having a size between 10,000 bp and 99,999 bp; and d. a fourth plasmid DNA molecule having a size between 100,000 bp and 300,000 bp.

In various embodiments, the assessment kit further comprises an exonuclease, wherein the exonuclease is configured to digest linear polynucleotides but does not digest circular DNA.

In various embodiments, the assessment kit further comprises a forward primer and a reverse primer for each of the first plasmid DNA molecule and the one or more of the second, the third, and the fourth plasmid DNA molecules.

In various embodiments, the first plasmid DNA molecule is linearized pEGFP-C1, or wherein the first plasmid DNA molecule is pEGFP-C1 and the restriction endonucleases comprise BamHI and EcoRI.

In various embodiments, the second plasmid DNA molecule is pCMV-Cre, the third plasmid DNA molecule is a fosmid, and the fourth plasmid DNA molecule is a bacterial artificial chromosome (BAC) clone.

In various embodiments, the exonuclease comprises exonuclease V, Plasmid-Safe™ ATP-Dependent DNase, or both.

In various embodiments, the forward primer and the reverse primer is selected from those disclosed in Table 2.

Various embodiments of the invention provide for a kit for isolation of extrachromosomal circular DNA (eccDNA) from cells or tissues and assessment of the eccDNA, the kit comprising: an alkaline buffer for cell lysis; a proteinase and/or an RNase; an anion-exchange column or agarose; a substantially neutral buffer for recovery of covalently closed circular DNA isolated with the anion-exchange column; an exonuclease, which does not digest circular DNA; and the contents of the assessment kit for use with a sample containing circular DNA as described herein.

Various embodiments of the invention provide for a method for enriching and quantifying extrachromosomal circular DNA (eccDNA) in a cell or tissue, wherein the cell or tissue comprises chromosomal DNA and the eccDNA, and the eccDNA comprises mitochondrial DNA and non-organelle eccDNA, the method comprising: lysing the cell or tissue, or obtaining a sample wherein the cell or tissue is lysed; and adding to the lysed cell or tissue, or to the sample: a first plasmid DNA molecule which is linearized, and one or more of: a second plasmid DNA molecule having a size between 1,000 bp and 9,999 bp; a third plasmid DNA molecule having a size between 10,000 bp and 99,999 bp; and a fourth plasmid DNA molecule having a size between 100,000 bp and 300,000 bp; to obtain a plasmid-spiked sample; treating the plasmid-spiked sample with a proteinase to degrade protein from the plasmid-spiked sample, and subsequently treating with an exonuclease to digest linear polynucleotides but not circular DNA, followed by recovery of DNA to obtain the eccDNA; and (iii) quantifying respective amounts of the first plasmid DNA molecule and the one or more of the second, the third, and the fourth plasmid DNA molecules after the treatment with the exonuclease; and/or quantifying an amount of the mitochondrial DNA and an amount of the chromosomal DNA.

In various embodiments, the method does not include multiple displacement amplification or rolling circle amplification of the cell or tissue, or the sample wherein the cell or tissue is lysed has not been amplified.

In various embodiments, the eccDNA comprises eccDNA of at least 10 kilobases (kb) in size and eccDNA between 0.5 kb and 10 kb.

In various embodiments, the cell or tissue is a cancerous cell or a tumor tissue.

In various embodiments, the method is for enriching and quantifying eccDNA in two or more cells or tissues, wherein a first cell or tissue is a cancerous cell or a tumor tissue, and the second cell or tissue is a normal cell or normal tissue obtained adjacent to the cancerous cell or the tumor tissue.

In various embodiments, the quantification comprises measuring a nucleic acid amount via one or more of quantitative polymerase chain reaction (PCR), next-generation sequencing, and Southern blotting.

In various embodiments, the quantification further comprises calculating a ratio or relative quantity (RQ) of a target circular DNA over a linear DNA in step (iii); and the target circular DNA is any one of the one or more of the second, the third, and the fourth plasmid DNA molecule, and the linear DNA is the first plasmid DNA molecule; or the target circular DNA is the mitochondrial DNA, and the linear DNA is a locus of the chromosomal DNA.

In various embodiments, ratio of the target DNA over the linear DNA is greater than 1000, thereby indicating the enrichment of the eccDNA.

In various embodiments, the linear DNA is chromosome 17 (chr17), and the target circular DNA is chromosome M (chrM).

In various embodiments, the first plasmid DNA molecule is linearized pEGFP-C1, the second plasmid DNA molecule is pCMV-Cre, the third plasmid DNA molecule is a fosmid, and the fourth plasmid DNA molecule is a bacterial artificial chromosome (BAC) clone.

In various embodiments, in step (i) lysing the cell or tissue comprises incubating the cell or tissue in a lysis buffer; and step (ii) further comprises treating the plasmid-spiked sample with an RNase, and treating the plasmid-spiked sample with an alkaline buffer following the treatment with the RNase.

In various embodiments, the lysis buffer comprises Tris-HCl, EDTA, SDS and NaCl; the treatment with the proteinase comprises adding proteinase K and incubating at about 50° C. for about 2 hours; the treatment with the RNase comprises adding RNase A and incubating at about 50° C. for about 1 hour; and the treatment with the alkaline buffer comprises adding a solution containing NaOH and SDS and incubating for about 5 minutes; and subsequent to step (ii) the method further comprises recovering the eccDNA from the plasmid-spiked sample following the treatment with the exonuclease by using a buffer exchange column, or centrifugal unit.

Various embodiments provide for a method for enriching and quantifying extrachromosomal circular DNA (eccDNA) in a cell or tissue, wherein the cell or tissue comprises chromosomal DNA and the eccDNA, and the eccDNA comprises mitochondrial DNA and non-organelle eccDNA, the method comprising: lysing the cell or tissue, or obtaining a sample wherein the cell or tissue is lysed; and adding to the lysed cell or tissue, or to the sample: a first plasmid DNA molecule which is linearized, and one or more of: a second plasmid DNA molecule having a size between 1,000 bp and 9,999 bp; a third plasmid DNA molecule having a size between 10,000 bp and 99,999 bp; and a fourth plasmid DNA molecule having a size between 100,000 bp and 300,000 bp, to obtain a plasmid-spiked sample; embedding the plasmid-spiked sample into agarose; digesting the agarose with b-agarese I; removing any remaining carbohydrate (if any); precipitating DNA to obtain eccDNA; and quantifying respective amounts of the first plasmid DNA molecule and the one or more of the second, the third, and the fourth plasmid DNA molecules; and/or quantifying an amount of the mitochondrial DNA and an amount of the chromosomal DNA.

In various embodiments, the method further comprises treating the plasmid-spiked sample with a proteinase to degrade protein from the plasmid-spiked sample, and subsequently treating with an exonuclease to digest linear polynucleotides but not circular DNA, before precipitating the DNA.

In various embodiments, the method does not include multiple displacement amplification or rolling circle amplification of the cell or tissue, or the sample wherein the cell or tissue is lysed has not been amplified.

In various embodiments, the eccDNA comprises eccDNA of at least 100 kilobases (kb), or at least 150 kb in size.

In various embodiments, the cell or tissue is a cancerous cell or a tumor tissue.

In various embodiments, the method is for enriching and quantifying eccDNA in two or more cells or tissues, wherein a first cell or tissue is a cancerous cell or a tumor tissue, and the second cell or tissue is a normal cell or normal tissue obtained adjacent to the cancerous cell or the tumor tissue.

In various embodiments, the quantification comprises measuring a nucleic acid amount via one or more of quantitative polymerase chain reaction (PCR), next-generation sequencing, and Southern blotting.

In various embodiments, the quantification further comprises calculating a ratio or relative quantity (RQ) of a target circular DNA over a linear DNA;

and the target circular DNA is any one of the one or more of the second, the third, and the fourth plasmid DNA molecule, and the linear DNA is the first plasmid DNA molecule; or the target circular DNA is the mitochondrial DNA, and the linear DNA is a locus of the chromosomal DNA.

In various embodiments, the ratio of the target DNA over the linear DNA is greater than 1000, thereby indicating the enrichment of the eccDNA.

In various embodiments, the linear DNA is chromosome 17 (chr17), and the target circular DNA is chromosome M (chrM).

In various embodiments, the first plasmid DNA molecule is linearized pEGFP-C1, the second plasmid DNA molecule is pCMV-Cre, the third plasmid DNA molecule is a fosmid, and the fourth plasmid DNA molecule is a bacterial artificial chromosome (BAC) clone.

In various embodiments, in step (i) lysing the cell or tissue comprises incubating the cell or tissue in a lysis buffer; and step (ii) further comprises treating the plasmid-spiked sample with an RNase, and treating the plasmid-spiked sample with an alkaline buffer following the treatment with the RNase.

In various embodiments, the lysis buffer comprises Tris-HCl, EDTA, SDS and NaCl; the treatment with the proteinase comprises adding proteinase K and incubating at about 50° C. for about 2 hours; the treatment with the RNase comprises adding RNase A and incubating at about 50° C. for about 1 hour; and the treatment with the alkaline buffer comprises adding a solution containing NaOH and SDS and incubating for about 5 minutes; and subsequent to step (ii) the method further comprises recovering the eccDNA from the plasmid-spiked sample following the treatment with the exonuclease by using a buffer exchange column, centrifugal unit, or an agarose plug.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1E depict purification and enrichment of naïve small circular DNA (nscDNA) without in vitro amplification. FIG. 1A depicts a schematic overview for the purification and enrichment of nscDNA. Cell lysis was followed by circular DNA isolation through an anion-exchange resin typically used for isolating plasmids. Contaminating linear DNA was digested by Plasmid-Safe DNase (Lucigen) (or by exonuclease V (New England Biolab)). Following qPCR to confirm enrichment of exonuclease-resistant nscDNA, nscDNA libraries were constructed, sequenced, and analyzed through our NGS pipeline. gDNA samples in which nscDNA was not enriched were also analyzed. FIG. 1B depicts the qPCR results displaying the mtDNA:chr17 ratio (log scale) of enriched nscDNA from cell lines preceding library construction. Relative quantities for both targets were normalized to gDNA samples. N=1 for all gDNA. N=3 for Colo320DM/Hela S3 nscDNA. N=2 for IMR-90/GM12878 nscDNA. qPCR reactions were run in quadruplicates. Data represents the mean+SEM. FIG. 1C depicts that 60 ng of HeLa S3 gDNA and nscDNA (both sonicated by Bioruptor Pico for 0s/15s/45s) were size-fractionated by PFGE (left), stained with SYBR Gold, and analyzed by Southern blotting with hybridization to a DIG-labeled mtDNA probe (right). FIG. 1D depicts the percentages of paired reads in nscDNA from cell lines mapped to chrM. N=1 for all gDNA. N=3 for Colo320DM/HeLa S3 nscDNA. N=2 for IMR-90/GM12878 nscDNA. Data represents the mean+SD. FIG. 1E depicts Read coverages per 1 kb bin (the number of reads divided by per-million scaling factor) of the gDNA (blue) and nscDNA (yellow/orange) for the amplified DM locus in Colo320DM (top) and for the chromosomally integrated amplicons in the sister cell line, Colo320HSR (bottom). The DM locus in chr8 is displayed, along with 500 kb surrounding regions.

FIGS. 1F-1I depict the characteristics of cell line-derived nscDNA. FIG. 1F depicts the qPCR results displaying the relative quantity (normalized to gDNA) of mtDNA and chr17 targets in HeLa S3 nscDNA digested with either Plasmid-Safe DNase (nscDNA-PS) or Exonuclease V (nscDNA-EV). N=1 for nscDNA-PS/nscDNA-EV. qPCR reactions were run in quadruplicates. Data represents the mean+SEM. FIG. 1G depicts the percentages of paired reads in HeLa S3 nscDNA-PS/nscDNA-EV mapped to chrM. N=1 for nscDNA-PS/nscDNA-EV. FIG. 1H depicts the qPCR analysis of the relative quantity of mtDNA (top) and chr17 (bottom) DNA targets in HeLa S3 nscDNA (blue) or RCA-amplified nscDNA (green). Relative quantities for both targets were normalized to nscDNA samples without RCA (no RCA). Three independent RCA reactions were conducted for each of the experiments. qPCR reactions were run in quadruplicates. Data represents the mean+SEM. FIG. 1I depicts an ELISA-based method was used to compare LINE-1 global DNA methylation in GM12878/HeLa S3 nscDNA (yellow) and gDNA (salmon) samples. In vitro amplified DNA does not retain methylation and the samples amplified by RCA are presented as negative controls. The relative methylation of the samples is displayed normalized to gDNA, with P-values (two-tailed test) comparing nscDNA to in vitro amplified nscDNA for both GM12878 (P=2.6×10−6) and HeLa S3 (P=0.003). N=2 for all samples. Data represents the mean+SD.

FIGS. 2A-2G depict exemplary genomic, molecular, and cytogenetic analysis of cell line-derived nscDNA. FIG. 2A depicts the percentages of total mappable unique (blue) and multi-mapped (yellow) reads in gDNA and nscDNA to hg38, excluding reads mapped to chrM. N=1 for all gDNA. N=3 for Colo320DM/HeLa S3 nscDNA. N=2 for IMR-90/GM12878 nscDNA. Data represents the mean+SD. FIG. 2B depicts the percentages of total mappable reads aligned to repetitive elements (SINE/LINE/Simple/Satellite) in gDNA and nscDNA as defined by RepeatMasker in hg38, excluding reads mapped to chrM. N=1 for all gDNA. N=3 for Colo320DM/HeLa S3 nscDNA. N=2 for IMR-90/GM12878 nscDNA. Data represents the mean+SD. FIG. 2C depicts the geneious software snapshot of GM12878 nscDNA coverage peaks (blue) found in chr10q11.21 (top) and chr7p 11.2 (bottom) after paired read alignment to hg38. The repetitive elements and SDs associated with these regions are displayed using the UCSC Genome Browser. Light to dark gray and light to dark yellow SD tracks indicate 90-98% and 98-99% sequence similarity between SDs, respectively. FIG. 2D depicts the percentages of total mappable reads in gDNA and nscDNA reads aligned to SD tracks in hg38. Reads mapped to SD tracks in mtDNA were excluded. N=1 for all gDNA. N=3 for Colo320DM/HeLa S3 nscDNA. N=2 for IMR-90/GM12878 nscDNA. Data represents the mean+SD. FIG. 2E depicts that 60 ng of HeLa S3 gDNA and nscDNA (both sonicated by Bioruptor Pico for Os/15s/45s) were analyzed by Southern blotting with hybridization to DIG-labeled SD probes. The associated agarose gel is displayed in FIG. 1C. FIG. 2F depicts the representative metaphase FISH spreads of HeLa S3 chromosomes stained with DAPI (blue) and hybridized with two different SD-harboring fosmid clones, G248P81785G8 (top) and G248P86779H3 (bottom), as probes. The probes were labeled with two different haptens, DIG (red) or Biotin (green). Yellow signals (yellow arrows) in which probes co-localized were considered positive. White arrows indicate localization of only one probe. All scale bars are equal to 10 μm. FIG. 2G depicts the number of co-localized extrachromosomal signals in HeLa S3 metaphase spreads with two SD-harboring probes (G248P81785G8/G248P86779H3). The number of spreads (y-axis) and the number of signals (0-5) counted (x-axis) are displayed. A probe (RP11-685G23) representing a single-copy genomic region was used as a negative control. The error bars represent 95% confidence intervals. P-values (Fisher's exact test, two-tailed) were calculated comparing two outcomes (signal vs. no signal) for G248P81785G8 (P=0.0001) and G248P86779H3 (P=0.0003) against RP11-685G23.

FIGS. 2H-2M depict the repetitive elements and segmental duplications in cell line-derived nscDNA. FIG. 2H depicts the percentages of total gDNA and nscDNA reads that remained unmapped to hg38. N=1 for all gDNA. N=3 for Colo320DM/HeLa S3 nscDNA. N=2 for IMR- 90/GM12878 nscDNA. Data represents the mean+SD. FIG. 2I depicts the percentages of total mappable gDNA and nscDNA reads aligned to SINE (green), LINE (blue), simple (gold), and satellite (gray) repetitive elements, excluding mtDNA reads. N=1 for all gDNA. N=3 for Colo320DM/HeLa S3 nscDNA. N=2 for IMR-90/GM12878 nscDNA. Data represents the mean+SD. FIG. 2J depicts the SD contents within chrM (1-16,569 bp in hg38) are displayed from the UCSC Genome Browser. Light to dark gray and light to dark yellow SD tracks indicate 90-98% and 98-99% sequence similarity between SDs, respectively. FIG. 2K depicts the percentages of gDNA and nscDNA reads mapped to SD tracks in hg38. Reads mapped to SD tracks in mtDNA were included, which is different from FIG. 2D. N=1 for all gDNA. N=3 for Colo320DM/HeLa S3 nscDNA. N=2 for IMR- 90/GM12878 nscDNA. Data represents the mean+SD. FIG. 2L depicts the representative metaphase-FISH spread of GM12878 chromosomes stained with DAPI (blue) and hybridized with a SD-harboring fosmid clone G248P86779H3 as a probe. The probe was labeled with two different haptens, DIG (red) and Biotin (green). Yellow signals (yellow arrows) in which probes co-localized were considered positive. White arrows indicate localization of only one probe. All scale bars are equal to 10 μm. FIG. 2M depicts the number of co-localized extrachromosomal signals in 25 GM12878 metaphase spreads were counted after hybridization to SD-harboring probes (G248P81785G8/G248P86779H3) or a probe (RP11-685G23) representing a single-copy genomic region as a negative control. The number of spreads and the number of signals (0-5) counted are displayed on the y-axis and x-axis, respectively. The error bars represent 95% confidence intervals. P-values (Fisher's exact test, two-tailed) were calculated comparing two outcomes (signal vs. no signal) for G248P81785G8 (P=0.0001) and G248P86779H3 (P=0.0009) against RP11-685G23.

FIG. 3A-3H depict the genomic compositions of sperm nscDNA. FIG. 3A depicts qPCR results of the pUC18:pEGFP-C1 ratio (log 10 scale) of enriched human/mouse sperm nscDNA preceding library construction. During sperm lysis, nscDNA and gDNA sample preparations were spiked with equal amounts of circular pUC18 and linearized pEGFP-C1 (EcoRI-BamHI). Relative quantities for both targets were normalized to gDNA samples. N=1 for human sperm gDNA. N=2 for human/mouse sperm nscDNA and mouse sperm gDNA. aPCR reactions were run in quadruplicates. Data represents the mean+SEM. FIG. 3B depicts the percentages of total mappable unique (blue) and multi-mapped (yellow) reads in sperm gDNA and nscDNA aligned to hg38 or mm10. N=1 for human sperm gDNA. N=2 for human/mouse sperm nscDNA and mouse sperm gDNA. Data represents the mean+SD. FIG. 3C depicts the percentages of total mappable reads in sperm gDNA and nscDNA samples aligned to SINE (green), LINE (blue), simple (gold), and satellite (gray) repetitive elements. N=1 for human sperm gDNA. N=2 for human/mouse sperm nscDNA and mouse sperm gDNA. Data represents the mean+SD. FIG. 3D depicts the percentages of total mappable sperm gDNA and nscDNA reads aligned to SD tracks in hg38/mm10. N=1 for human sperm gDNA. N=2 for human/mouse sperm nscDNA and mouse sperm gDNA. Data represents the mean+SD. FIG. 3E depicts an Integrative Genomics Viewer (IGV) snapshot of human sperm gDNA and nscDNA coverage peaks (blue) found in chr1 following alignment to hg38. The repetitive elements and SDs associated with this region are displayed using the UCSC Genome Browser. Light to dark gray SD tracks indicate 90-98% sequence similarity between SDs. FIG. 3F depicts the percentages of total mappable human sperm gDNA and nscDNA reads aligned to SD tracks in hg38, after removing the intersections between satellite repeats and SDs. N=1 for human sperm gDNA. N=2 for human sperm nscDNA. Data represents the mean+SD. FIG. 3G depicts the ratio of gDNA and nscDNA segmental duplication reads to uniquely mapped reads (MAPQ40). The fold changes in the nscDNA to gDNA ratios are indicated. N=1 for human sperm gDNA. N=2 for human/mouse sperm nscDNA and mouse sperm gDNA. Data represents the mean+SD. FIG. 3H depicts the percentages of total mapped reads in human sperm gDNA and nscDNA to hg38 (blue) or CHM13 (yellow) human reference genomes. N=1 for human sperm gDNA. N=2 for human sperm nscDNA. Data represents the mean+SD.

FIGS. 31 and 3J depict molecular analysis of germline-derived nscDNA. FIG. 3I are transmission electron microscope images of human sperm nscDNA (left panels) and gDNA (right panels). Scale bar=150 nm. FIG. 3J depicts that 30 ng of human sperm gDNA and nscDNA (both sonicated by Bioruptor Pico for Os/45s) were size-fractionated by PFGE, stained with SYBR Gold, and analyzed by Southern blotting with hybridization to DIG-labeled SD probes (left) or a satellite probe (right).

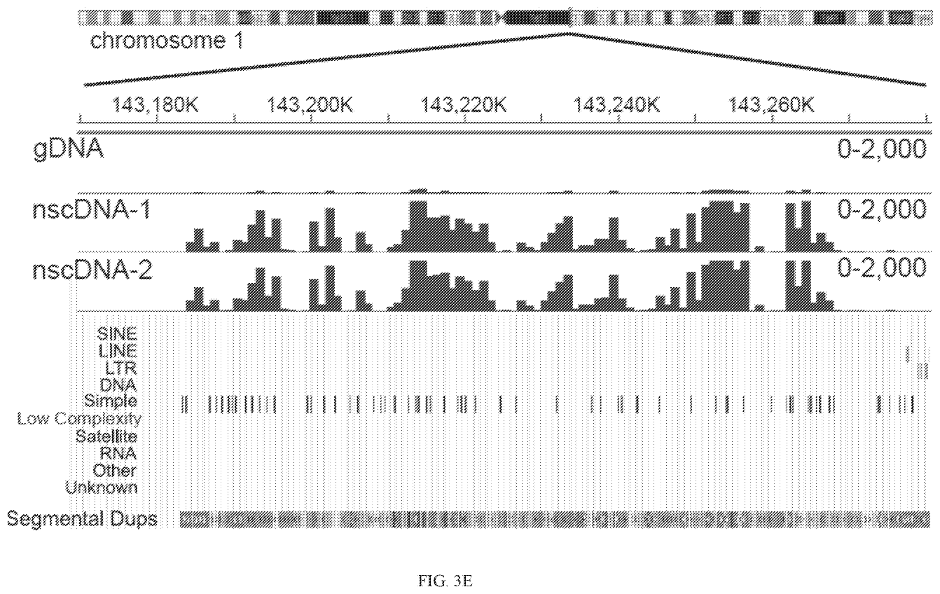

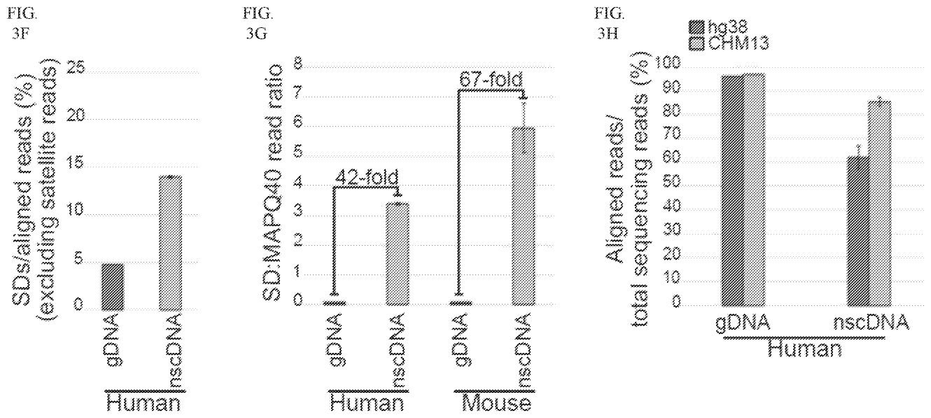

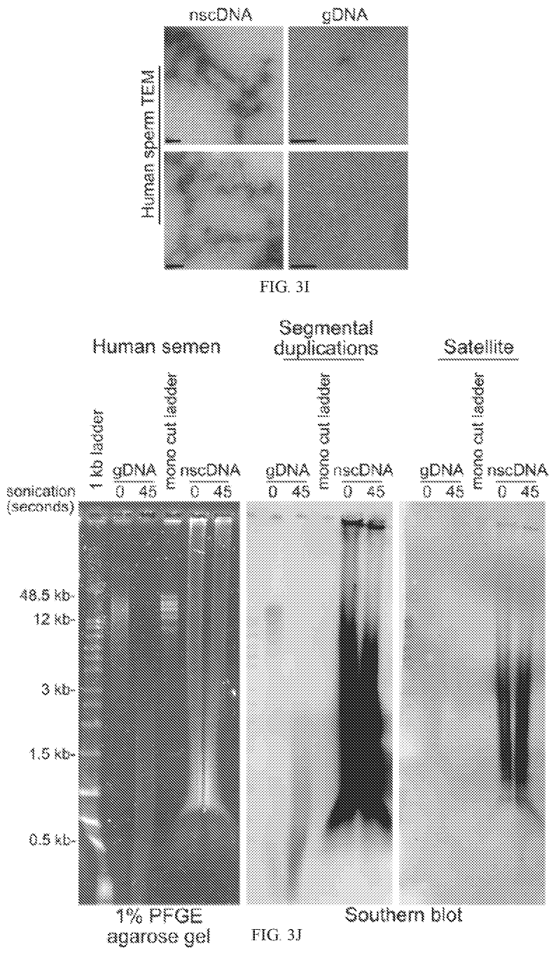

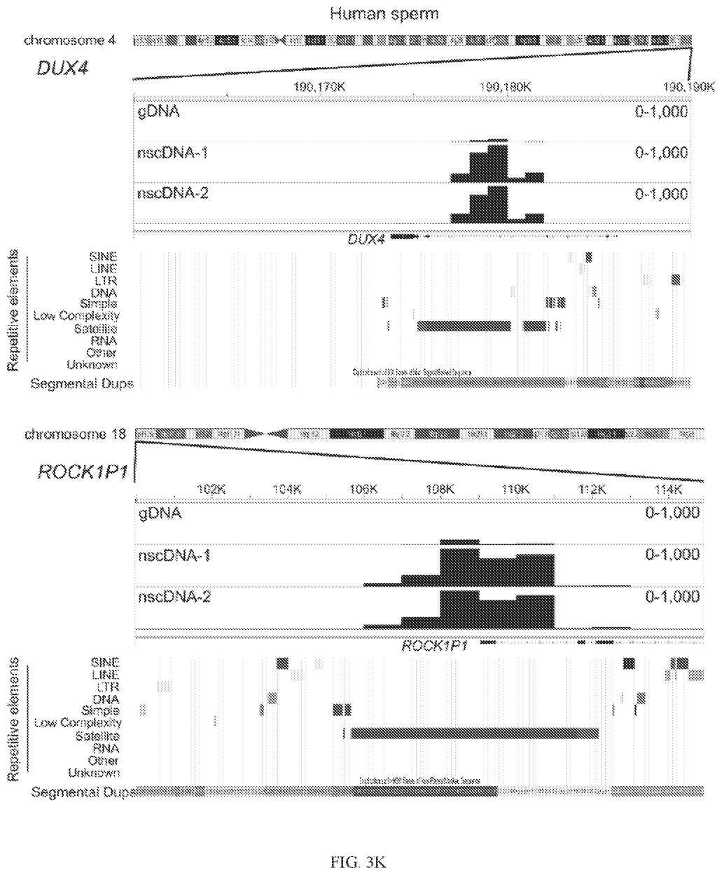

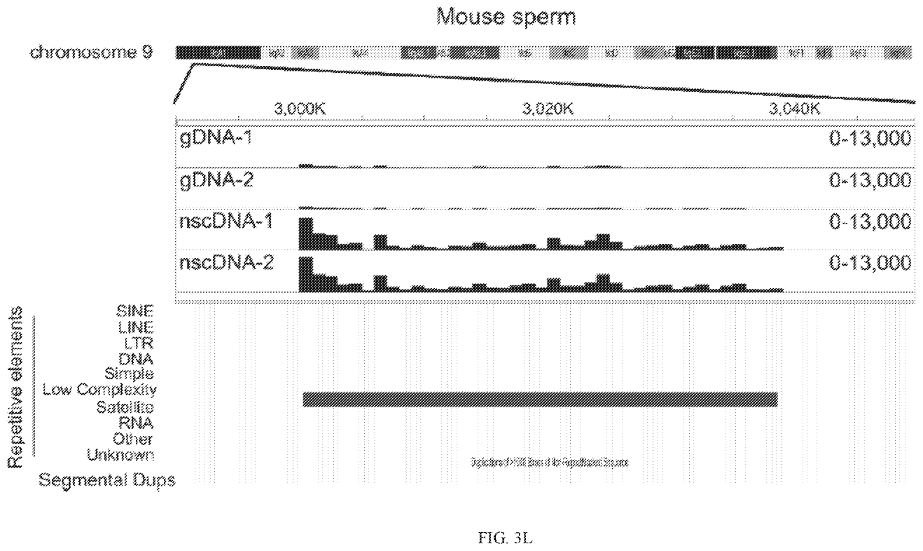

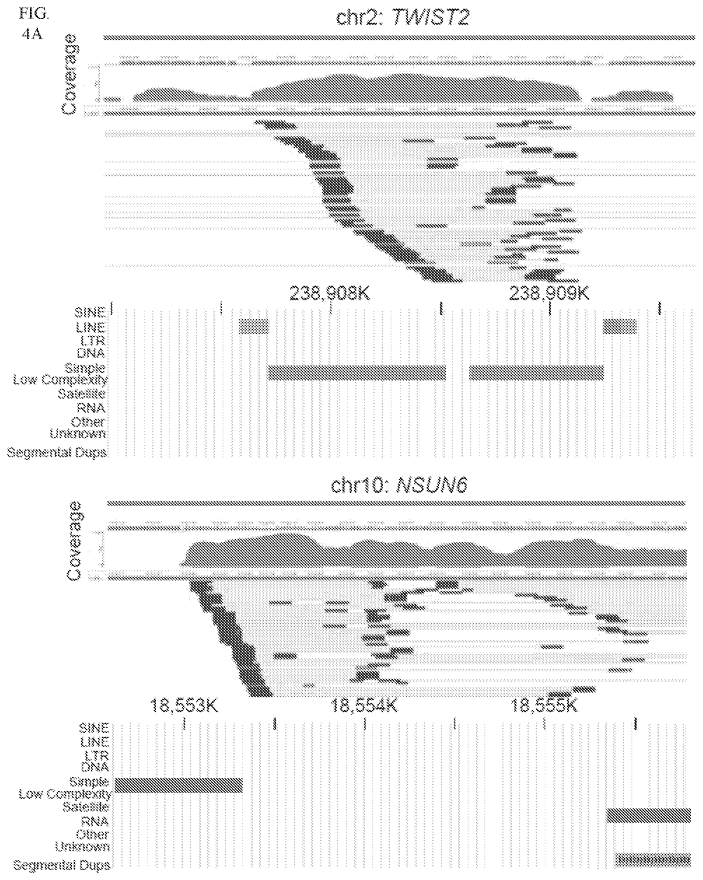

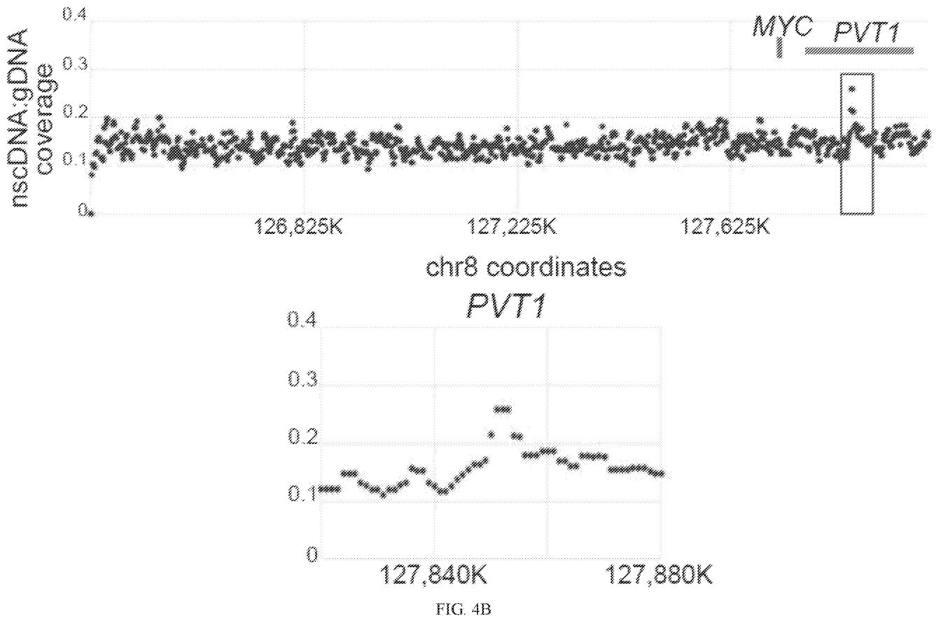

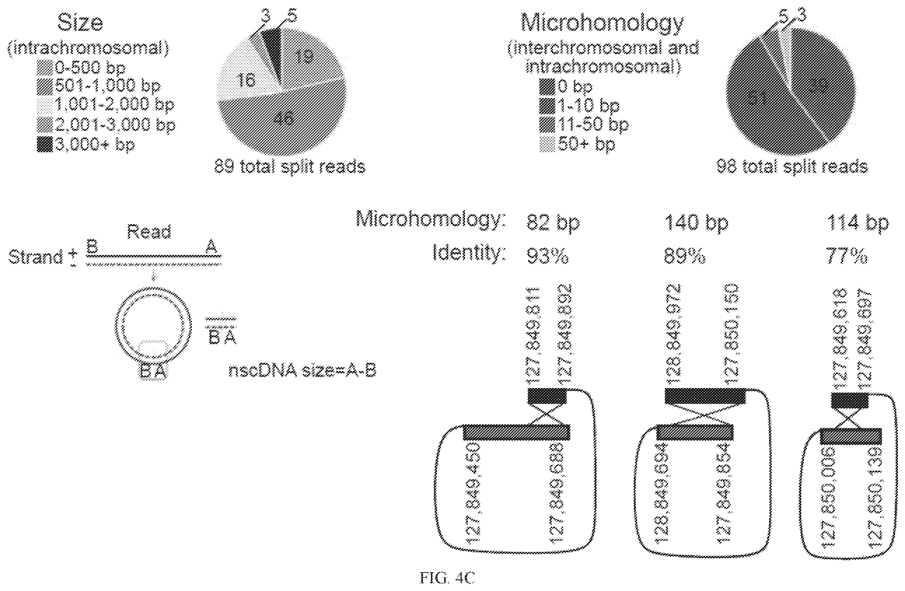

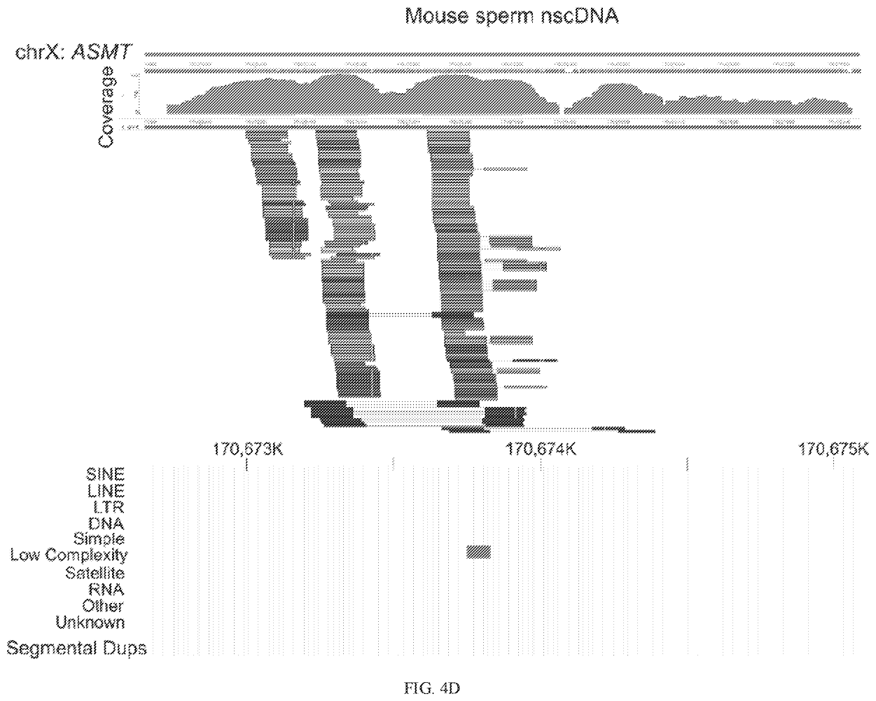

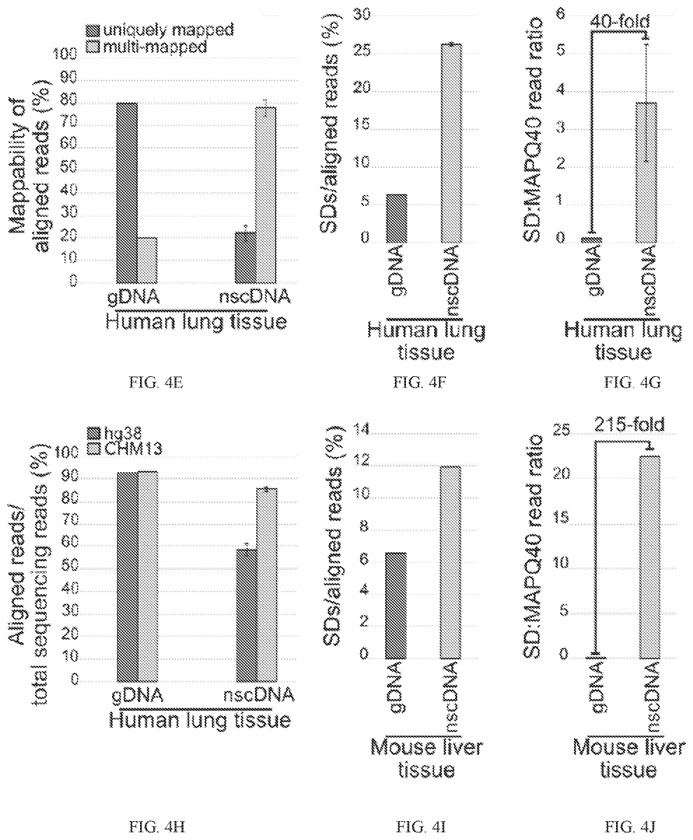

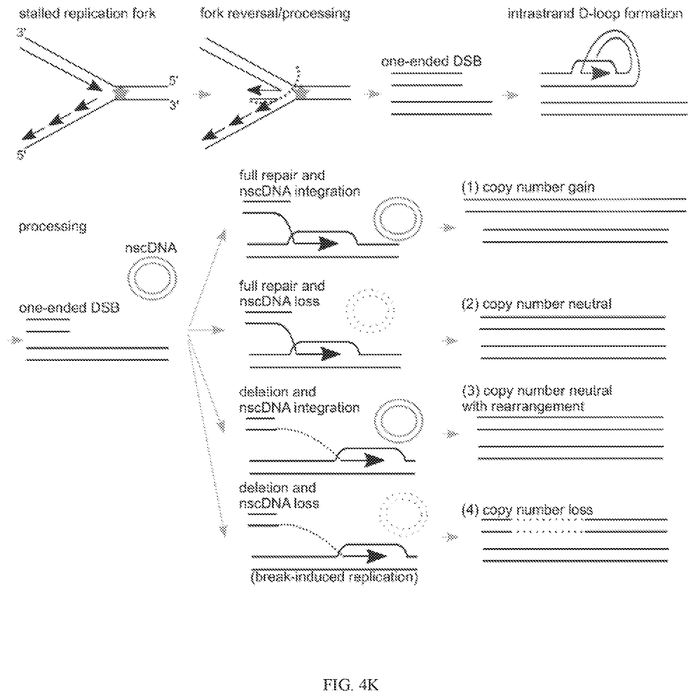

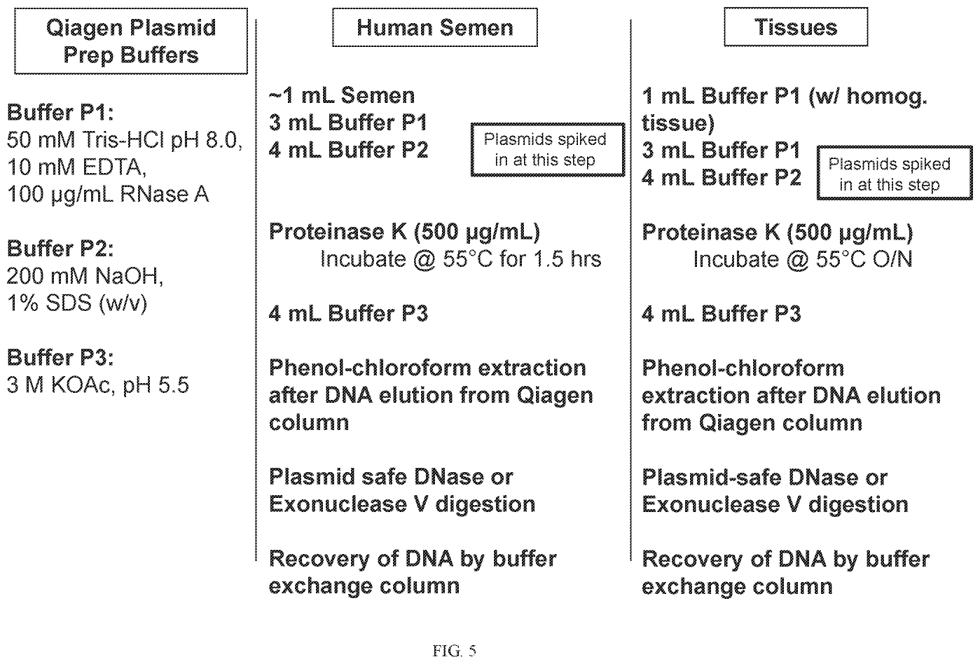

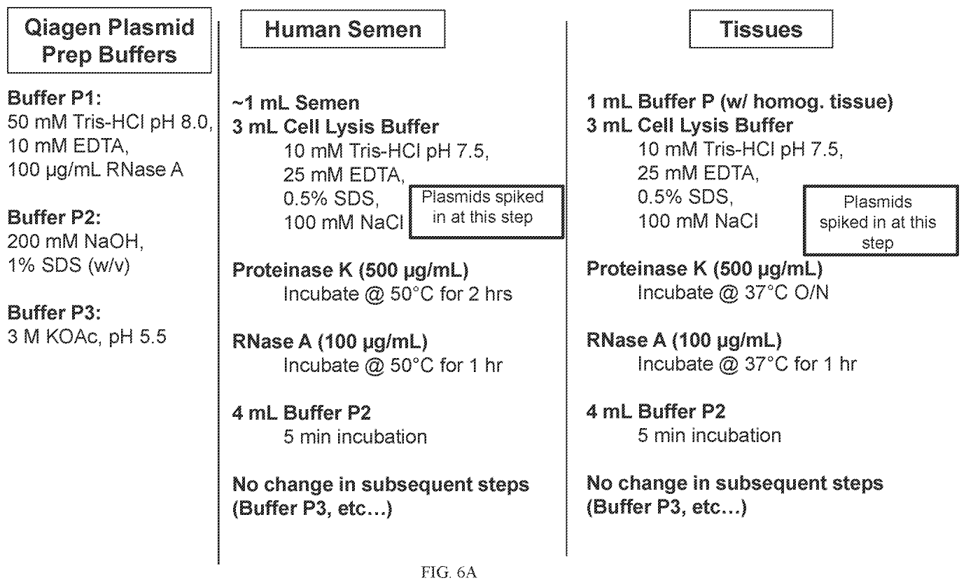

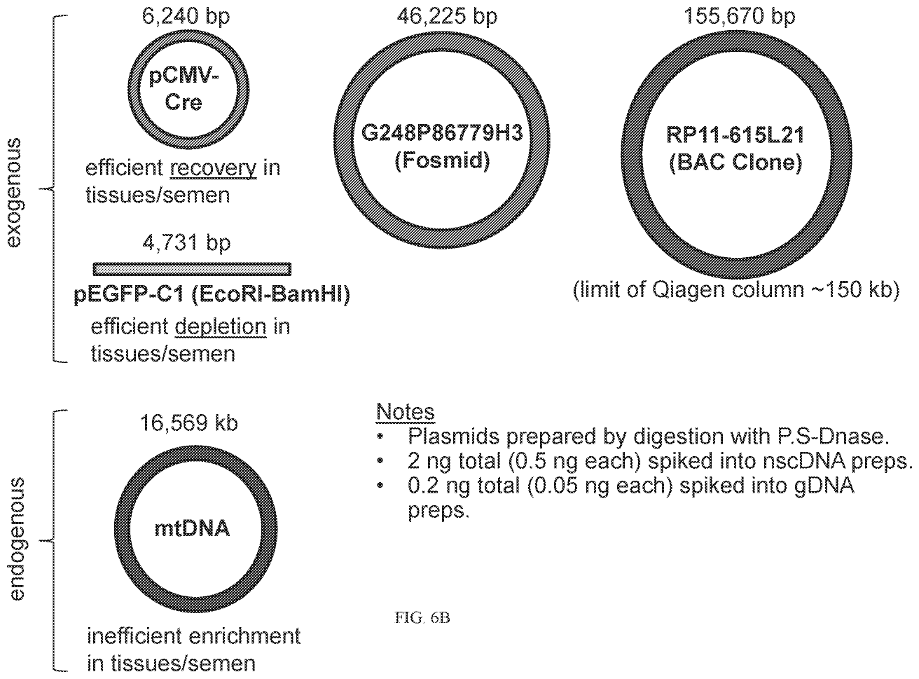

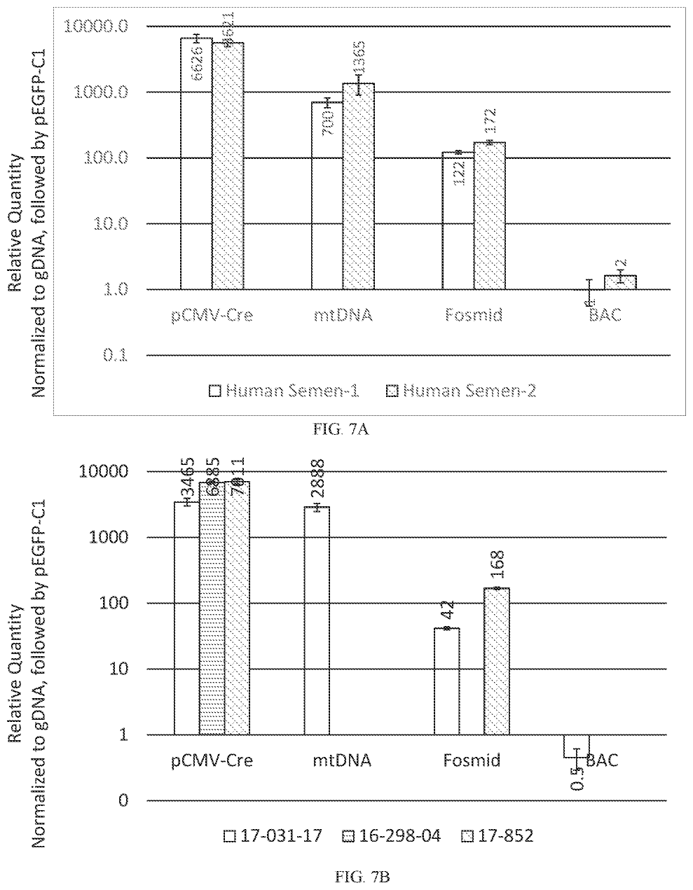

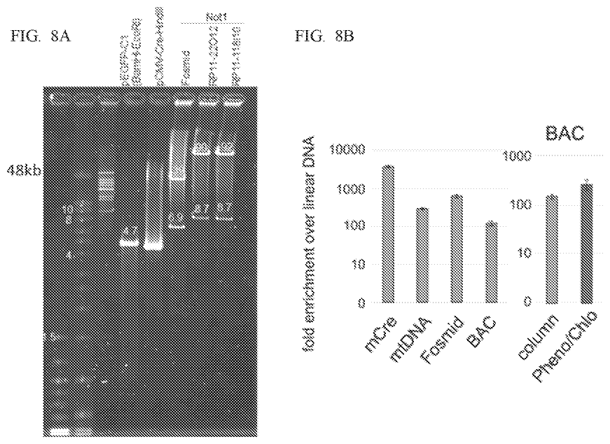

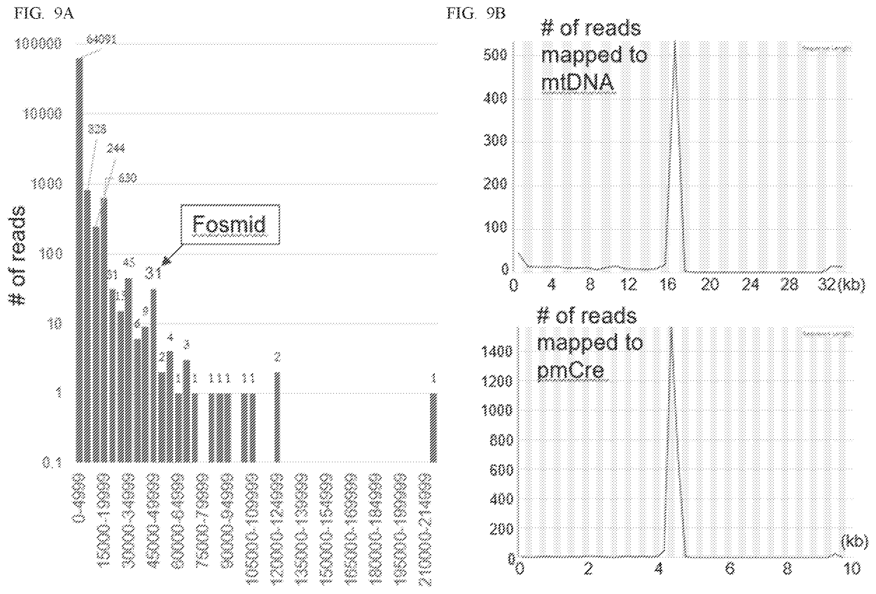

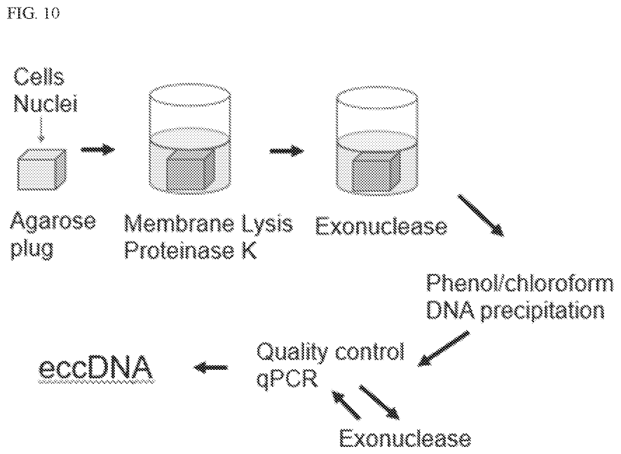

FIGS. 3K and 3L depict peaks overlapping genes, SDs, and repetitive elements in germline-derived nscDNA. FIG. 3K depicts IGV snapshots of human sperm gDNA and nscDNA coverage peaks (blue) overlapping genes, SDs, and satellite elements in chr4 and chr18 following alignment to hg38. The repetitive elements and SDs associated with these regions are displayed using the UCSC Genome Browser. The y-axis shows the coverage data range (0-1,000) for all samples. FIG. 3L depicts an IGV snapshot of mouse sperm gDNA and nscDNA coverage peaks (blue) found in chr9 following alignment to mm10. The repetitive elements associated with this region is displayed using the UCSC Genome Browser. The y-axis shows the coverage data range (0-13,000) for all samples.

FIGS. 4A-4C depict fusion junctions in nscDNA. FIG. 4A depicts outward facing paired reads are an indication of circular DNA and cover the fusion point of circles. In the circular orientation, paired reads (red arrows) are facing inward and straddle the fusion point. After fragmentation, library construction, and sequencing, the two reads in the pair map to distant locations in the opposite, outward orientation linked by a red dashed line on hg38. Representative examples of coverage peaks (blue) with linked outward-facing paired reads (red boxes linked by a solid red line) in human sperm nscDNA (hg38 alignment, MAPQ40 reads) are shown for the NSUN6 gene region on chr10 and the TWIST2 gene region on chr2 using Geneious software. The repetitive element and SD contents in these regions are displayed using the UCSC Genome Browser. Light to dark gray SD tracks indicate 90-98% sequence similarity between SDs. FIG. 4B depicts the coverage per 1 kb bin (with 5 bin median filter) of the amplified DM (126,425,000-128,000,000 kb) locus in Colo320DM nscDNA after normalization to gDNA coverage is shown. A coverage peak consisting of six consecutive bins (127,849,000-127,855,000 kb) at the PVT1 locus is shown. FIG. 4C depicts individual reads from Colo320DM nscDNA that partially mapped to the PVT1 locus in chr8 (hg38) were identified and analyzed for fusion junctions. Junction coordinates were used to calculate the potential sizes of nscDNA. Microhomologies were calculated by nucleotide overlaps at the fusion points, with the longest homologies depicted (82 bp, 114 bp, and 140 bp). Junctions between different chromosomes were excluded from the size analysis.

FIG. 4D depicts mouse sperm nscDNA fusion points. A representative example of a read coverage peak (blue) with linked outward-facing paired reads (red boxes linked by a solid red line) in mouse sperm nscDNA-1 (mm10 alignment, MAPQ40 reads). The ASMT gene region on chrX is displayed using Geneious software.

FIGS. 4E-4J depict overrepresentation of segmental duplications in tissue-derived nscDNA. FIG. 4E depicts the percentages of total mappable unique (blue) and multi-mapped (yellow) reads in gDNA and nscDNA from human lung tissue aligned to hg38. N=1 for gDNA and N=2 for nscDNA. Data represents the mean+SD. FIG. 4F depicts the percentages of total mappable reads aligned to SD tracks in hg38 from human lung tissue gDNA (blue) and nscDNA (yellow). N=1 for gDNA and N=2 for nscDNA. Data represents the mean+SD. FIG. 4G depicts the ratio of human lung tissue gDNA (blue) and nscDNA (yellow) segmental duplication reads to uniquely mapped reads (MAPQ40). The fold change in the nscDNA to gDNA ratio is indicated. N=1 for gDNA and N=2 for nscDNA. Data represents the mean+SD. FIG. 4H depicts the percentages of total mapped reads in human lung tissue gDNA and nscDNA to hg38 (blue) or CHM13 (yellow) human reference genomes. N=1 for gDNA and N=2 for nscDNA. Data represents the mean+SD. FIG. 4I depicts the percentages of total mappable mouse liver tissue gDNA (blue) and nscDNA (yellow) reads aligned to SD tracks in mm10. N=1 for gDNA and nscDNA. Data represents the mean. FIG. 4J depicts the ratio of mouse liver tissue gDNA (blue) and nscDNA (yellow) segmental duplication reads to uniquely mapped reads (MAPQ40). The fold change in the nscDNA to gDNA ratio is indicated. N=1 for gDNA and nscDNA. Data represents the mean.

FIG. 4K depicts a replication-dependent model for the mechanism underlying the formation of nscDNA and copy number variation. A replication fork stalls within a region of duplicated DNA. Fork reversal creates a four-way junction, the processing of which generates a chromatid with a one-ended double-strand break (DSB) and an intact chromatid. DSB invasion into the same chromatid results in the formation of nscDNA and another chromatid with a one-ended DSB. The invasion of the one-ended DSB into the sister chromatid, possibly by break-induced replication, would fully recover DNA content (outcomes 1-3). The integration of nscDNA results in copy number gain (outcome and the loss of nscDNA leads to a copy number neutral state (outcomes 2 and 3). One ended DSBs could invade into an ectopic locus, and, with the loss of nscDNA, lead to copy number loss (outcome 4). Another potential outcome would be that the ectopic integration of nscDNA could recover the DNA contents at the cost of genetic rearrangement.

FIG. 5 depicts an overview of the procedure and reagents used in Example 1 to extract/isolate eccDNA from human semen or from a tissue.

FIG. 6A depicts an overview of a modified procedure (compared to FIG. 5) and its reagents used in Example 3 to extract/isolate eccDNA from human semen or from a tissue. FIG. 6B depicts exemplary ‘control’ DNAs for assessment or validation of (non-organelle) eccDNA, wherein some are exogenously added linearized plasmid DNA and circular plasmid DNA molecules, and another can be endogenously present mtDNA (circular).

FIG. 7A depicts qPCR results (presented in Log 10 scale) of circular ‘control’ DNAs in a procedure according to FIG. 6A (after proteinase K and exonuclease V digestions) to isolate eccDNA from two exemplary human semen samples. FIG. 7B depicts qPCR results (presented in Log 10 scale) of circular ‘control’ DNAs in a procedure according to FIG. 6A (after proteinase K and exonuclease V digestions) to isolate eccDNA from three exemplary human breast tumor tissue samples.

FIG. 8A-8B depicts control circular DNA clones. 8A. Each clone was digested with restriction enzymes to confirm the sizes. 8B. right, the fold enrichments of control clones and mtDNA, measured by qPCR. (left) Fold enrichment of 102 kb BAC clone was tested for two independent eccDNA recovery methods.

FIG. 9A depicts size distribution of 65,949 reads. FIG. 9B depicts peaks for mtDNA and pmCre at expected sizes (x-axis). y-axis, the number of reads.

FIG. 10 depicts exemplary procedures. Cells or nuclei from solid tissue will be mounted in agarose plug. After membrane lysis, the plug will be treated with exonuclease. After DNA recovery, the enrichment of control clones will be tested by qPCR. If enrichment is not sufficient, exonuclease treatment will be repeated.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

The term “covalently closed circular DNA” refers to DNA molecules that have assumed a circular form in contrast to linear DNA molecules such as eukaryotic chromosomal DNA or bacterial chromosomal DNA that comprises a nick or comprises a free 3′ or 5′ end. Moreover, the circular structure of the above referenced DNA molecules is covalently closed. ecc DNA is known in the art and is further described, for example, in K. G. Hardy (ed.), Plasmid, a Practical Approach (IRL Press Oxford U.K., Washington D.C., U.S.A., 1987).

Methods are herein provided to assess or quantify isolated eccDNA from a cell, a tissue, or body fluid, and optimally the isolated eccDNA is validated through assessment or quantification in the methods to be essentially free of linear, genomic DNA after the isolation. The term “essentially free of linear, genomic DNA” is intended to mean that more than 95% of the linear, genomic DNA, preferably more than 98%, and most preferably more than 99% of the linear, genomic DNA in a sample has been degraded and/or removed. In a general term, genome refers to the complete set of genes or genetic material present in a cell or organism; and cells have linear and circular DNA. Optimally, the eccDNA isolated according to the invention is 100% purified or is as close to 100% purified as is within the limits of detection using standard assays.

“DNA spiking” herein relates to “plasmid spiking,” and it refers to a spiked control, which is when plasmid (or some synthetic DNA with a specific known sequence) is added to a sample that will provide some signal to a reaction (herein, exonuclease digestion). This procedure can be used to discover if the exonuclease treatment to degrade linear DNA molecules (such as eukaryotic chromosomal DNA) in the isolation of eccDNA is working correctly—for validation purpose, and to quantify the extent of this degradation, which includes comparing the amount of linear DNA (or the relative linear DNA amount to circular DNA amount) before and after the exonuclease treatment.

Extrachromosomal circular DNA (eccDNA) originates from linear chromosomal DNA in various human tissues under physiological and disease conditions. The genomic origins of eccDNA have largely been investigated using in vitro amplified DNA. However, in vitro amplification precludes the quantitative assessment of eccDNA by skewing the total population stoichiometry and the absolute quantity. In addition, the analyses have focused on eccDNA stemming from single-copy genomic regions, leaving a comprehensive picture of nuclear origins undetermined. To overcome these issues and gain a broader scope of eccDNA characteristics, we developed a strategy to enrich endogenous eccDNA populations at the native state, without RCA, which we designated as naïve small circular DNA (nscDNA). The purity of nscDNA populations was rigorously validated by both internal (mitochondrial DNA) and exogenous (plasmid) controls. We integrated molecular, cytogenetic and genomic approaches to examine the sizes, physical locations, and nature of nscDNA. In contrast to previous studies that infer eccDNA lengths based on genomic locations of putative circular DNA, we directly measured the size distribution of nscDNA populations with Southern blotting. We found that human nscDNA ranges from approximately 0.5 to greater than 10 kb, encompassing relatively unexplored size populations of circular DNA between microDNA (<400 bp) and DMs.

We also rigorously examined genomic origins of nscDNA beyond single-copy regions and found that nscDNA is predominantly derived from duplicated sequences of the human genome, including segmental duplications (SDs). SDs represent recently duplicated genomic regions and are considered to be actively evolving areas of the human genome, as they are strongly associated with copy number variations and contribute more to genetic diversity in humans than single nucleotide polymorphisms. The strong association of duplicated sequences with genetic diversity prompted us to study germline-derived nscDNA in human and mouse sperm and tissues, which we found to be overrepresented with SDs.

Methods for enriching, quantifying, assessing, and/or validating the presence or enrichment of, extrachromosomal circular DNA (eccDNA) are provided. In various aspects, the methods are for enriching, quantifying, and/or validating the presence or enrichment of eccDNA in a cell or tissue or cell culture, wherein the cell or tissue comprises chromosomal DNA and the eccDNA, and the eccDNA comprises mitochondrial DNA and non-organelle eccDNA. In some aspects, the methods are for assessing enrichment of eccDNA extracted from tissues and/or body fluids, such as normal tissue or cancerous tissues. In various aspects, the methods include measuring or quantifying one or both of: a ratio (or relative amount) of endogenous mitochondrial DNA (mtDNA) to chromosomal DNA (linear), and a ratio (or relative amount) of an exogenously added, circular plasmid DNA to another exogenously added, linearized plasmid DNA, said endogenous referring to originally present in the cell or tissue and said exogenous referring to adding substances not originally present in the cell or tissue, wherein the measurement is after an exonuclease treatment to digest linear DNA in the cell or tissue. In some aspects, a high ratio (e.g., ≥1,500, ≥1,000, ≥900, ≥800, ≥600, ≥500, ≥400, ≥200, or ≥100; e.g., ratio of the log 10 scale amounts) of the mtDNA amount to the chromosomal DNA amount following the exonuclease digestion, indicates an enrichment or validation of extraction of the eccDNA. In some aspects, due to the exonuclease's digestion of linear DNA but not circular DNA, the ratio (e.g., the ratio of the numbers of DNA copies) of the exogenously added circular plasmid DNA to the exogenously added linearized plasmid DNA becomes much larger after the exonuclease digestion, compared to the ratio initially added (a known proportion); and so an increase in this ratio (e.g., ≥1,500-fold, ≥1,000-fold, ≥900-fold, ≥800-fold, ≥600-fold, ≥500-fold, ≥400-fold, ≥200-fold, ≥100-fold, or ≥50-fold) indicates a successful digestion of background linear DNA, and therefore enrichment or validation of extracted eccDNA.

In some embodiments, a method for enriching, assessing, or quantifying isolated DNA (e.g., circular DNA, eccDNA, or in some instances genomic DNA) from a cell or tissue includes the steps of:
(i) lysing the cell or tissue, or obtaining a sample wherein the cell or tissue is lysed; and
(ii) (A)adding to the lysed cell or tissue, or to the sample:
   a. a first plasmid DNA molecule which is linearized, and
      one or more of:
   b. a second plasmid DNA molecule which is circular and having a size between 1,000 bp and 9,999 bp;
   c. a third plasmid DNA molecule which is circular and having a size between 10,000 bp and 99,999 bp; and
   d. a fourth plasmid DNA molecule which is circular and having a size between 100,000 bp and 300,000 bp;
   to obtain a plasmid-spiked sample;
      treating the plasmid-spiked sample with a proteinase (e.g., proteinase K) and/or an RNase (preferably a proteinase and an RNase, either concurrently or sequentially) to degrade protein and RNA, followed by DNA recovery via a circular DNA-enriching column (QIAGEN);
      and
      (B) treating the plasmid-spiked sample with an exonuclease (e.g., exonuclease V, an ATP-dependent DNase) to digest linear polynucleotides but not circular DNA.

In some embodiments, a method for enriching, assessing, or quantifying isolated DNA (e.g., circular DNA, eccDNA, or in some instances genomic DNA) from a cell lysate includes the steps of:
(A) adding to the cell lysate:
   c. a first plasmid DNA molecule which is linearized, and
      one or more of:
   d. a second plasmid DNA molecule which is circular and having a size between 1,000 bp and 9,999 bp;
   c. a third plasmid DNA molecule which is circular and having a size between 10,000 bp and 99,999 bp; and
   d. a fourth plasmid DNA molecule which is circular and having a size between 100,000 bp and 300,000 bp;
   to obtain a plasmid-spiked sample; and
   treating the cell lysate with a proteinase (e.g., proteinase K) and an RNase to degrade protein and RNA in the cell lysate, followed by passing the proteinase- and RNase-treated cell lysate through a circular DNA enriching column to recover DNA from the cell lysate; and subsequently:
(B) treating the product after step (A) with an exonuclease (e.g., exonuclease V, an ATP-dependent DNase) to hydrolyze/digest linear double-stranded polynucleotides but not circular DNA.

In some aspects of step (A), the addition of plasmids takes place before the treatment with proteinase and/or RNase. In other aspects of step (A), the addition of plasmids occurs after the treatment with proteinase and/or RNase.

In further aspects, following exonuclease treatment in step (B), the circular DNA is recovered by a buffer exchange column to remove residue exonuclease and buffer from previous steps, or recovered and concentrated using a centrifugal filter (e.g., DNA Fast Flow PCR Grade centrifugal filters by MICROCON®, e.g., MRCFOR100ET).

In various aspects, the method further includes measuring the amount of the first plasmid DNA molecule and that of the one or more of the second, the third, and the fourth plasmid DNA molecules after the treatment with the exonuclease; and/or measuring an amount of the mitochondrial DNA and an amount of the chromosomal DNA. In further aspects, the method includes calculating a ratio of the circular DNA amount to the linear DNA amount, wherein the circular DNA refers to the one or more of the second, the third, and the fourth plasmid DNA molecules or the mitochondrial DNA, and the linear DNA refers to the first plasmid DNA molecule or the chromosomal DNA; and comparing the ratio after the exonuclease treatment to the ratio before the exonuclease treatment, or obtaining a higher ratio after the exonuclease treatment relative to the ratio before the exonuclease treatment.

In various embodiments, the methods disclosed herein for enriching, assessing, or quantifying eccDNA does not include multiple displacement amplification or rolling circle amplification of the cell or tissue before or during the steps, or the sample wherein the cell or tissue is lysed has not been amplified.

In some embodiments, the eccDNA is isolated or extracted using an existing isolation protocol or kit (e.g., QIAGEN's Plasmid Prep kit/buffers, OMEGABIOTEK's EZNA® Plasmid DNA kit, or traditional boiling-lysis or alkaline lysis), and the eccDNA of various sizes can be enriched and assessed/quantified according to the methods disclosed herein at least, including but not limited to eccDNA of greater than 10 kilobases (kb) in size, greater than 50 kb, greater than 100 kb, between 1 kb and 10 kb, between 10 kb and 50 kb, between 50 kb and 100 kb, between 100 kb and 200 kb, between 1 kb and 100 kb, or greater than 0.5 kb.

In some embodiments, the quantification comprises measuring a nucleic acid amount via one or more of quantitative polymerase chain reaction (qPCR), next-generation sequencing, and Southern blotting.

In some embodiments, the following are denoted as ‘control’ DNAs used for quantifying the enrichment or presence of eccDNA, and ‘control’ DNA includes circular DNAs such as the (exogenously added) one or more of the second, the third, and the fourth plasmid DNA molecules, as well as the (endogenously present) mitochondrial DNA, and the linear DNAs such as the (exogenously added) first plasmid DNA molecule which is linearized, as well as the (endogenously present) chromosomal DNA.

In some aspects, the ‘control’ DNAs for the method include a linear DNA which is chromosome 17 (chr17) and a circular DNA which is chromosome M (chrM). Other exemplary linear DNA include 18S.

In some aspects, the ‘control’ DNAs include the first plasmid DNA molecule which is linearized pEGFP-C1, and one or more of circular plasmid DNA molecules such as pCMV-Cre (about 6,240 bp), a fosmid (such as G248P86779H3 of about 46,225 bp), and a bacterial artificial chromosome (BAC) clone (such as RP11-615L21 of about 155,670 bp).

In some embodiments of the methods, lysing the cell or tissue comprises mixing the cell or tissue in a lysis buffer with exogenously added ‘control’ DNAs, wherein the lysis buffer can be an aqueous solution of Tris-HCl, EDTA, SDS and NaCl; and treating this mixture by incubating it with an exonuclease (e.g., proteinase K, or PLASMID-SAFE™ ATP-Dependent DNase) at an appropriate temperature for an effective length of time to digest linear DNAs in the mixture; and followed by running the exonuclease-treated mixture onto an ion-exchange material for separation/collection/purification of circular DNA (including eccDNA in the lyzed cell/tissue).

In some aspects, an RNase solution is added initially along with the lysis buffer to the cell or tissue, before the exonuclease treatment. In other aspects, an RNase solution is added to the mixture after the exonuclease treatment, and before the quantitative PCR, sequencing or another measurement technique of the circular DNA. In some implementations, the spiked plasmids do no need to be removed. The remainder exogenously spiked DNA is estimated to make up no more than 10% of the isolated eccDNA. In some aspects, the exogenously spiked DNA makes up no more than 9%, 8%, 7%, 6%, 5%, 4%, 3%, 2%, or 1% (in weight or in molecule number) of the isolated eccDNA. The recovered eccDNA can be sequenced and mapped onto human or mouse or a respective genome (with a focus on the eccDNA derived from the human, the mouse, or the respective genome). In other implementations, following the exonuclease treatment, the remainder circular ‘control’ DNAs is removed from the collected eccDNA, for example, by use of appropriate endonucleases to digest or linearize the plasmid DNAs but not the cell-derived circular DNA.

A kit or a system is also provided. The kit can be an assessment kit for use with a sample containing circular DNA. In various embodiments, the kit or system comprises, or consists of, a linearized plasmid DNA molecule and one or more circular plasmid DNA molecules, of a known or predetermined proportion. Preferably, the one or more circular plasmid DNA molecules collectively cover an approximate size range of eccDNA compositions in the sample, e.g., within one or two orders of magnitude larger or smaller than the largest, the median, and/or the smallest eccDNA composition.

In some embodiments, the kit includes:
a. a first plasmid DNA molecule which is linearized, or
   a first plasmid DNA molecule and one or more restriction endonucleases to linearize the first plasmid DNA; and one or more of:
b. a second plasmid DNA molecule which is circular and having a size between 1,000 bp and 9,999 bp;
c. a third plasmid DNA molecule which is circular and having a size between 10,000 bp and 99,999 bp; and
d. a fourth plasmid DNA molecule which is circular and having a size between 100,000 bp and 300,000 bp.

In some embodiments, the kit further includes an exonuclease. The exonuclease is configured to digest linear polynucleotides but does not digest circular DNA. Exemplary exonucleases are exonuclease V, or an ATP-dependent DNase under the tradename PLASMID-SAFE™.

In some embodiments, the kit further includes a forward primer and a reverse primer for each of the first plasmid DNA molecule and the one or more of the second, the third, and the fourth plasmid DNA molecules.

In some embodiments, the kit includes a first plasmid DNA molecule which is linearized pEGFP-C1, or the kit includes a first plasmid DNA molecule which is pEGFP-C1 and the restriction endonucleases, BamHI and EcoRI, such that the pEGFP-C1 can be treated with the restriction endonucleases by a user to be linearized.

In some embodiments, the kit includes a second plasmid DNA molecule which is pCMV-Cre, a third plasmid DNA molecule which is a fosmid, and/or a fourth plasmid DNA molecule which is a bacterial artificial chromosome (BAC) clone.

In some aspects, the kit includes one linearized plasmid DNA molecule and one circular plasmid DNA molecule. In some aspects, the kit includes one linearized plasmid DNA molecule and two circular plasmid DNA molecules. In some aspects, the kit includes one linearized plasmid DNA molecule and three circular plasmid DNA molecules. In some aspects, the linearized plasmid DNA molecule and the one or more circular plasmid DNA molecules are provided in an equal mass quantity in the kit, or are added in an equal mass quantity to the (lysed) cell or tissue, e.g., 0.5 ng each, about 0.1-0.3 ng each, about 0.3-0.7 ng each, or about 0.7-1 ng each to a cell/tissue sample in preparation for circular DNA; or about 0.05 ng each, about 0.01-0.03 ng each, about 0.30-0.07 ng each, or about 0.07-0.1 ng each to a cell/tissue sample in preparation for genomic DNA.

The forward primers and the reverse primers for some of the exemplary plasmid DNA molecules, and for an exemplary chromosomal DNA and exemplary mitochondrial DNA, are selected from those disclosed in Table 2.

Further embodiments provide the assessment kit in a preparation kit of eccDNA. In some embodiments, a kit is provided for isolation of extrachromosomal circular DNA (eccDNA) from cells or tissues and assessment of the eccDNA, and the kit includes an assessment kit disclosed herein; and one or more or all of:
a cell lysis buffer;
an alkaline buffer for DNA renaturation;
a proteinase and/or an RNase;
an anion-exchange material;
a substantially neutral buffer for recovery of covalently closed circular DNA isolated with the anion-exchange column;
an exonuclease;
a buffer exchange column (or centrifugal unit).

In some embodiments, a kit is provided for isolation of extrachromosomal circular DNA (eccDNA) from cells or tissues and assessment of the eccDNA, and the kit includes
an assessment kit disclosed herein; and
one or more or all of:
a cell lysis buffer;
an alkaline buffer for DNA renaturation;
a proteinase and/or an RNase;
agarose;
a substantially neutral buffer for recovery of covalently closed circular DNA isolated with the anion-exchange column;
an exonuclease;
a buffer exchange column (or centrifugal unit).

In various embodiments, the agarose is in the form of an agarose plug. In various embodiments, the agarose is low melting agarose. In various embodiments, low melting agarose melts at about 65.5° C. In various embodiments, low melting agarose melts at a temperature below the melting point of nucleic acids. In various embodiments, the agarose is 1% low melting agarose. In various embodiments, the agarose is about 0.5%-1.5% low melting agarose.

The Examples below describe exemplary reagents for components in a kit. Additional exemplary reagents and procedures for isolating or purifying circular DNA, or covalently closed circular DNA, are described in several publications such as U.S. Pat. No. 6,242,220, which is herein incorporated by reference in its entirety. Methods for the lysis of cells and unicellular organisms are described, for example, in Sambrook et al., Molecular Cloning, A Laboratory Handbook, 2nd edition (CSH Press, Cold Spring Harbor, U.S.A. 1989).

Exemplary plasmid DNA molecules for a kit or a method disclosed herein include but are not limited to pBR322, pGP564, pUG72, pUC19, and pSH63. Endonuclease cleavage sites are predetermined in known plasmids, so linearized plasmid DNA molecules can be prepared and provided. Additional examples of plasmids for spiking are described in publications such as Sims et al., J Mol Diagn., 18(3):336-349, 2016, which is herein incorporated by reference in its entirety.

Various embodiments of the invention provide for a method for enriching and quantifying extrachromosomal circular DNA (eccDNA) in a cell or tissue, wherein the cell or tissue comprises chromosomal DNA and the eccDNA, and the eccDNA comprises mitochondrial DNA and non-organelle eccDNA, the method comprising: lysing the cell or tissue, or obtaining a sample wherein the cell or tissue is lysed; and adding to the lysed cell or tissue, or to the sample: a first plasmid DNA molecule which is linearized, and one or more of: a second plasmid DNA molecule having a size between 1,000 bp and 9,999 bp; a third plasmid DNA molecule having a size between 10,000 bp and 99,999 bp; and a fourth plasmid DNA molecule having a size between 100,000 bp and 300,000 bp to obtain a plasmid-spiked sample; embedding the plasmid-spiked sample into agarose; digesting the agarose with b-agarese I; removing any remaining carbohydrate; precipitating DNA to obtain eccDNA; and quantifying respective amounts of the first plasmid DNA molecule and the one or more of the second, the third, and the fourth plasmid DNA molecules; and/or quantifying an amount of the mitochondrial DNA and an amount of the chromosomal DNA.

In various embodiments, the agarose is in the form of an agarose plug. In various embodiments, the agarose is low melting agarose. In various embodiments, the agarose is 1% low melting agarose. In various embodiments, the agarose is about 0/5%-1.5% low melting agarose.

In various embodiments, the method further comprises treating the plasmid-spiked sample with a proteinase to degrade protein from the plasmid-spiked sample, and subsequently treating with an exonuclease to digest linear polynucleotides but not circular DNA, before precipitating the DNA.

In various embodiments, the method does not include multiple displacement amplification or rolling circle amplification of the cell or tissue, or the sample wherein the cell or tissue is lysed has not been amplified.

In various embodiments, the eccDNA comprises eccDNA of at least 100 kilobases (kb) in size, or at least 150 kb in size.

In various embodiments, the cell or tissue is a cancerous cell or a tumor tissue.

In various embodiments, the method is for enriching and quantifying eccDNA in two or more cells or tissues, wherein a first cell or tissue is a cancerous cell or a tumor tissue, and the second cell or tissue is a normal cell or normal tissue obtained adjacent to the cancerous cell or the tumor tissue.

In various embodiments, the quantification comprises measuring a nucleic acid amount via one or more of quantitative polymerase chain reaction (PCR), next-generation sequencing, and Southern blotting.

In various embodiments, the quantification further comprises calculating a ratio or relative quantity (RQ) of a target circular DNA over a linear DNA;

and the target circular DNA is any one of the one or more of the second, the third, and the fourth plasmid DNA molecule, and the linear DNA is the first plasmid DNA molecule; or the target circular DNA is the mitochondrial DNA, and the linear DNA is a locus of the chromosomal DNA.

In various embodiments, the ratio of the target DNA over the linear DNA is greater than 1000, thereby indicating the enrichment of the eccDNA.

In various embodiments, the linear DNA is chromosome 17 (chr17), and the target circular DNA is chromosome M (chrM).

In various embodiments, the first plasmid DNA molecule is linearized pEGFP-C1, the second plasmid DNA molecule is pCMV-Cre, the third plasmid DNA molecule is a fosmid, and the fourth plasmid DNA molecule is a bacterial artificial chromosome (BAC) clone.

In various embodiments, in step (i) lysing the cell or tissue comprises incubating the cell or tissue in a lysis buffer; and step (ii) further comprises treating the plasmid-spiked sample with an RNase, and treating the plasmid-spiked sample with an alkaline buffer following the treatment with the RNase.

In various embodiments, the lysis buffer comprises Tris-HCl, EDTA, SDS and NaCl; the treatment with the proteinase comprises adding proteinase K and incubating at about 50° C. for about 2 hours; the treatment with the RNase comprises adding RNase A and incubating at about 50° C. for about 1 hour; and the treatment with the alkaline buffer comprises adding a solution containing NaOH and SDS and incubating for about 5 minutes; and subsequent to step (ii) the method further comprises recovering the eccDNA from the plasmid-spiked sample following the treatment with the exonuclease by using a buffer exchange column, centrifugal unit.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Quantitative Assessment Reveals the Dominance of Duplicated Sequences in Germline-Derived Extrachromosomal Circular DNA

Extrachromosomal circular DNA (eccDNA) plays a role in human diseases such as cancer, but little is known about the impact of eccDNA in healthy human biology. Since eccDNA is a tiny fraction of nuclear DNA, artificial amplification has been employed to increase eccDNA amounts, resulting in the loss of native compositions. We developed an approach to enrich eccDNA populations at the native state and captured a wide size distribution of populations that have not been extensively studied. Here, we report the quantitative assessment of naïve small circular DNA (nscDNA), obtained without in vitro amplification, by integrated genomic, molecular, and cytogenetic approaches. We found that, in human sperm, the vast majority of nscDNA came from high-copy genomic regions, including the most variable regions between individuals. nscDNA ranges up to tens of kilobases (kb) and is predominantly derived from multi-copy genomic regions, including segmental duplications (SDs). SDs, which account for 5% of the human genome and are hotspots of copy number variations, are significantly overrepresented in sperm nscDNA, with three times more sequencing reads derived from SDs than from the entire single-copy regions. SDs are also overrepresented in mouse sperm nscDNA, which we estimate to comprise 0.2% of nuclear DNA. Considering that eccDNA can shuttle between extra- and intra-chromosomal spaces, germline-derived nscDNA may be a mediator of genome diversity. Because eccDNA can be integrated back into chromosomes, eccDNA may promote human genetic variation.

Enrichment of eccDNA without In Vitro Amplification.

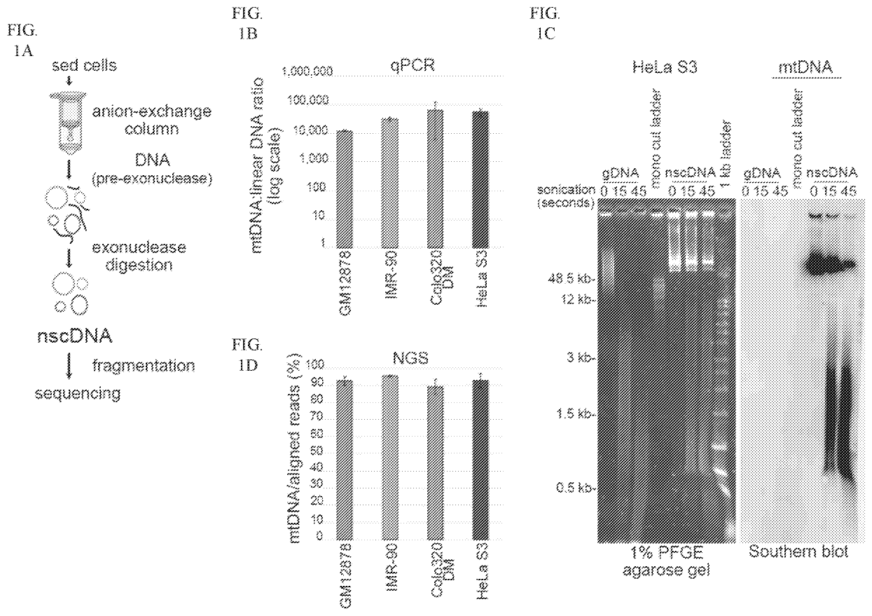

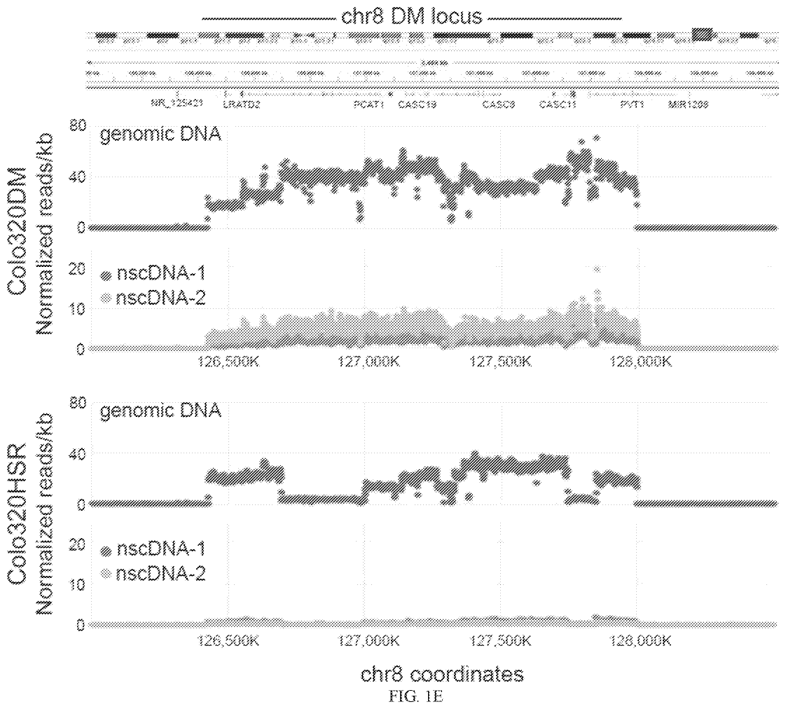

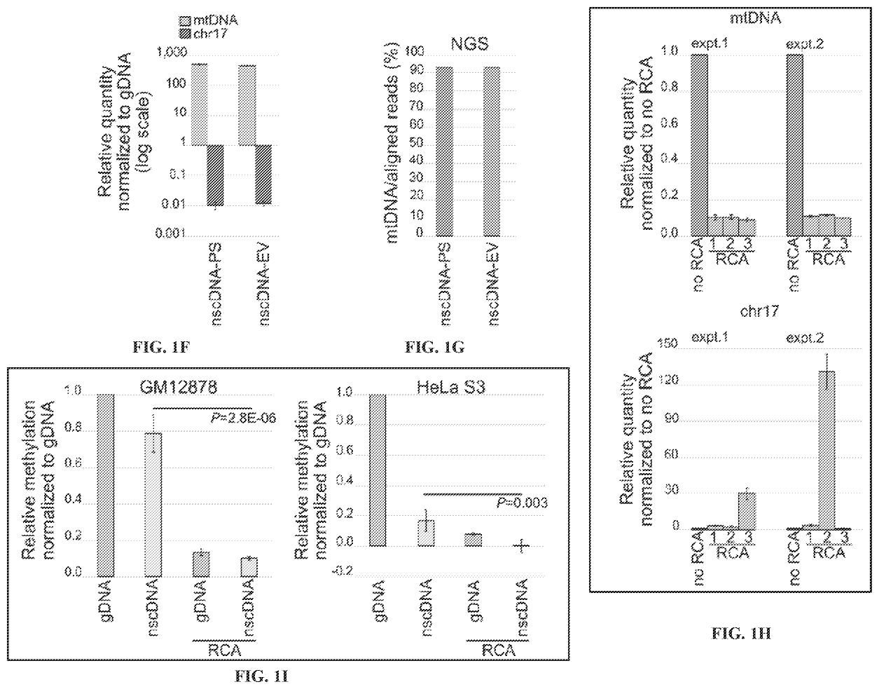

We developed a strategy to enrich nscDNA populations without in vitro amplification (FIG. 1A). In brief, we used alkaline conditions for cell lysis and neutral conditions to recover small circular DNA of covalently closed nature, which were captured through an anion-exchange column typically used for isolating bacterial plasmids. Following column purification, nscDNA-enriched samples were treated with excess amounts of exonuclease (Plasmid-Safe DNase), which efficiently hydrolyzes linear dsDNA. As a measure of nscDNA quality control, we analyzed the relative quantity of mitochondrial DNA (mtDNA) to chromosomal DNA after exonuclease digestion using quantitative PCR (qPCR) in human normal (IMR-90, GM12878) and cancer (HeLa S3, Colo320DM) cell lines (FIG. 1i). A representative locus of mtDNA, Chromosome M, served as an internal control for circular DNA, while a chr17 locus represented the population of linear DNA. The mtDNA:chr17 ratio, after normalization to genomic DNA (gDNA), was at least 10,000:1 in all cases, indicating extensive enrichment of circular DNA and depletion of linear DNA. The enrichment of mtDNA was reproducible when we used another commercially available exonuclease, E. coli Exonuclease V (Exo V) (FIG. 1F). gDNA was prepared from a replicate obtained from the same biological source as the nscDNA using a separate protocol to isolate total chromosomal DNA. gDNA is separately isolated, because it is not enriched with circular DNA, and therefore making it suitable for comparison with samples enriched with nscDNA. For the gDNA preparation, cells/homogenized tissue underwent cell lysis in a buffer (100 mM NaCl, 10 mM Tris, 25 mM EDTA, 0.5% SDS) with Proteinase K treatment overnight at 37° C. The gDNA was then purified by phenol/chloroform extraction and subsequent ethanol precipitation of DNA. gDNA samples were then spiked with equal amounts of the ‘control’ DNAs at this step for qPCR analysis. During qPCR, the gDNA samples were run in the same way as nscDNA. The RQ of mtDNA and chr17 were normalized to the same gDNA sample.

We further examined physical properties of HeLa S3 nscDNA by pulsed-field gel electrophoresis (PFGE) and sonication and found that nscDNA exhibited very distinct mobility and sonication response (FIG. 1C). A significant fraction of nscDNA was trapped in the wells, a characteristic of large circular DNA. Genomic DNA was very sensitive to sonication and high-molecular-weight linear DNA disappeared with 45 seconds of sonication. In contrast, nscDNA was relatively resistant to sonication, indicating another property of circular DNA. Finally, Southern blotting showed that mtDNA was extremely enriched in nscDNA over gDNA samples.

The enrichment of mtDNA in our nscDNA pools indicated the efficient recovery of circular DNA of large sizes (>10 kb), including size populations of eccDNA that have not been rigorously investigated. We sequenced cell line-derived nscDNA and aligned the reads to hg38 to investigate their compositions.

Specifically, gDNA and nscDNA were sequenced on the platforms. The reads were counted and mapped to the reference genome using bowtie 2 with unpaired alignment. The number of reads aligning more than once to the reference genome (aligned>1), only once to the reference genome (aligned=1), or not aligned (aligned=0) are reported by bowtie 2. The number of reads mapping to four types of repetitive elements (SINE/LINE/Simple/Satellite) were counted individually as well as using a combined track. The number of reads mapping in 1 kb non-overlapping bins was the control. In each case, reads mapping to chrM were not counted. The number of reads mapping to non-mitochondrial segmental duplications were counted. For the cell lines, regions on the nuclear chromosomes bearing high similarity to regions on chrM were excluded from the segmental duplication track. In contrast, for the human tissue and mouse samples, the entire chromosome was considered in the analyses.

In all nscDNA libraries, over 90% of reads mapped to chrM (FIG. 1D, FIG. 1G). The large extrachromosomal, double minute chromosomes (DMs) covering the 1.6-megabase region at 8q24.1 in Colo320DM provided an opportunity to test for the recovery of very large nscDNA of nuclear origin. At 8q24.1, nscDNA coverage increased compared to the 500 kb flanking regions on both sides (FIG. 1E). In contrast, nscDNA from Colo320HSR, a sister cell line with chromosomally integrated DMs, contained no notable increase in coverage, indicating the recovery of some megabase circular DNA through our procedure. Despite the overrepresentation, nscDNA coverage in Colo320DM was lower than gDNA coverage. Routine lab procedures, such as pipetting and DNA precipitation, shear large DNA molecules, and would render Mb circular DNA into linear DNA, which would be digested by exonuclease. Therefore, while circular DNA over 10 kb was efficiently enriched by our procedure, the recovery of Mb size circular DNA was limited.

A common method to enrich eccDNA involves RCA. However, RCA seemed to deplete large circular DNA and on occasion created artificial eccDNA. We compared HeLa S3 nscDNA pools amplified by RCA to non-amplified nscDNA and found that circular mtDNA quantities decreased 10-fold (FIG. 1H). We also observed that the copy number of chr17 was extremely higher in one of the replicates over others. Phi29 polymerase often switches templates. Switching templates within the same DNA fragment would initiate RCA and lead to amplification. Furthermore, while nscDNA retained DNA methylation, nscDNA amplified by RCA did not, as RCA copies methylated cytosine to cytosine. We measured the methylation of long interspersed nucleotide elements (LINE-1) in GM12878 and HeLa S3 DNA and found that amplified DNA exhibited a dramatic reduction of %5-methylcytosine to background levels, whereas non-amplified nscDNA retained methylation higher than background levels (FIG. 1I).

Over-Representation of Segmental Duplications in Cell Line-Derived nscDNA.

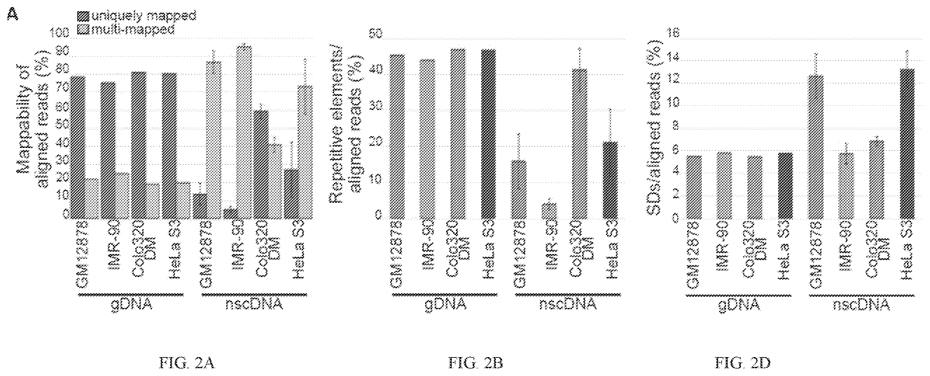

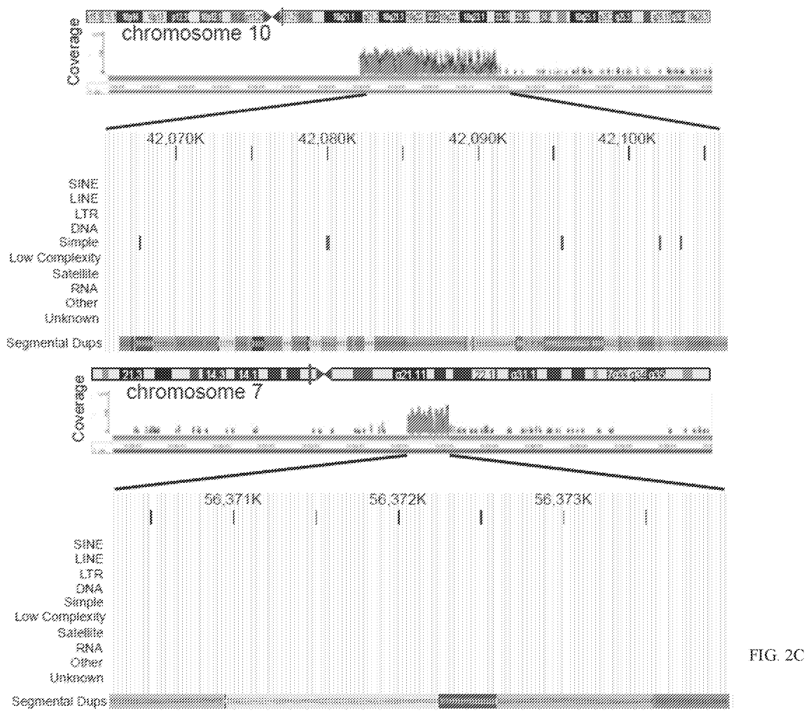

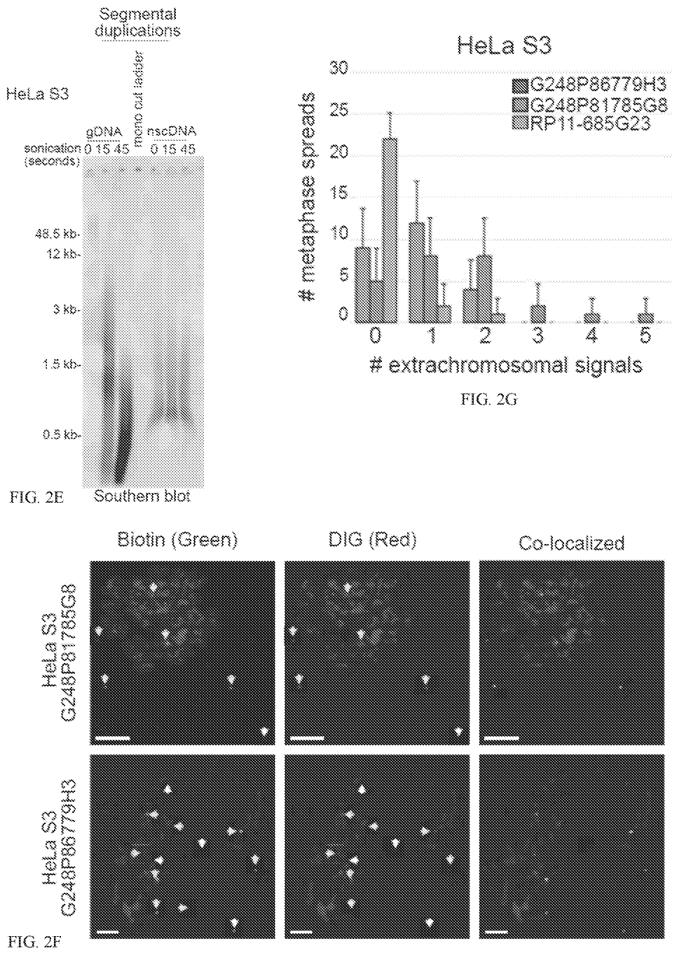

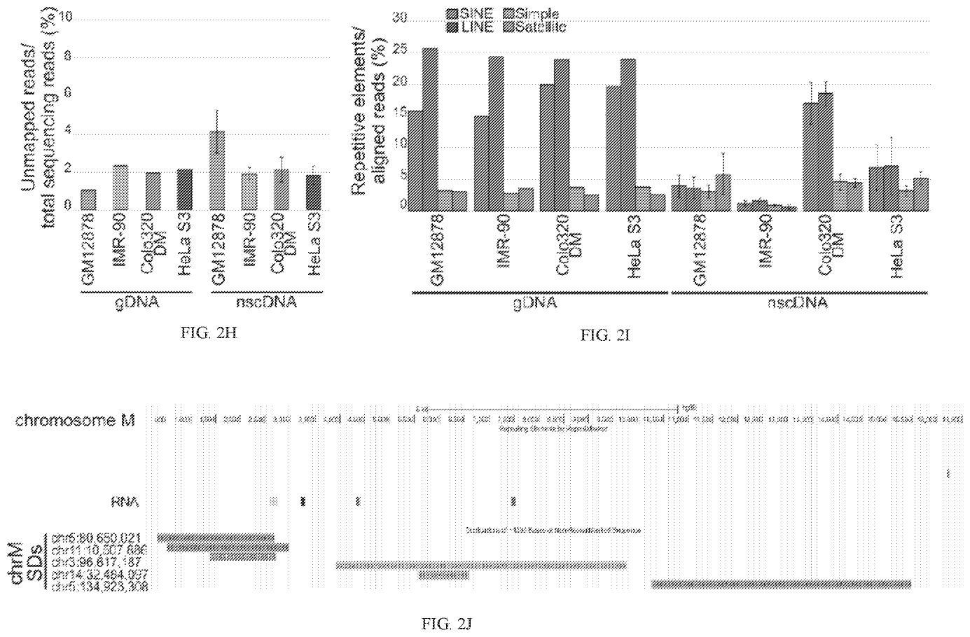

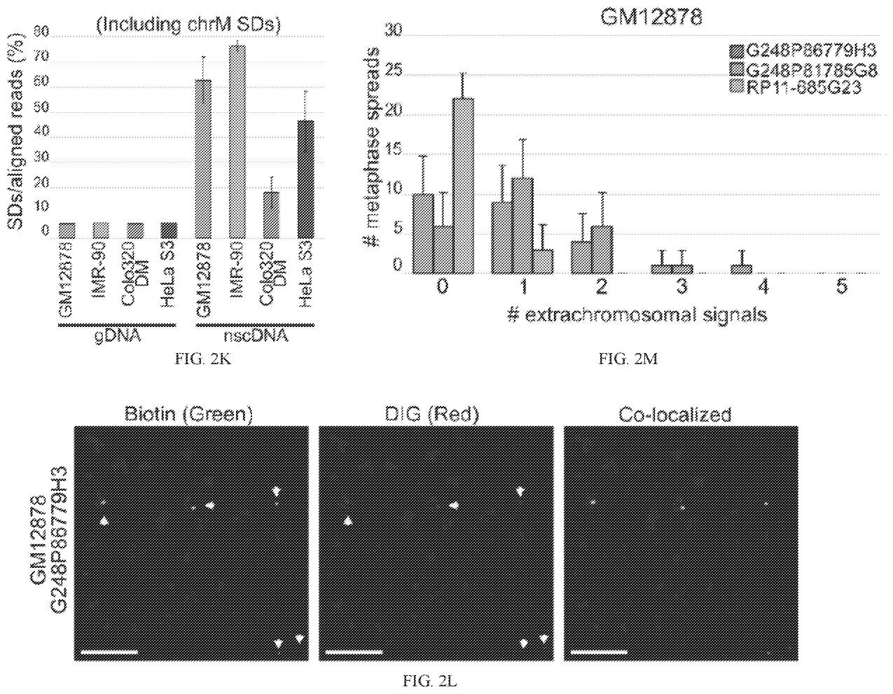

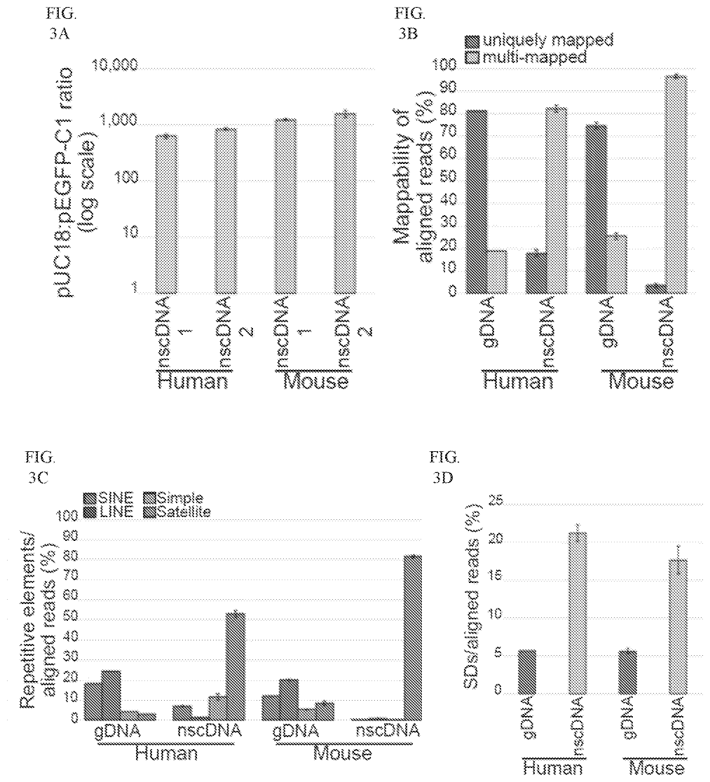

The very high fractions of mtDNA from cell lines limited the sequencing depth of nscDNA of nuclear origin; however, we proceeded to characterize the non-mtDNA reads using the bowtie2 aligner. We first confirmed the very high mappability (>95%) of nscDNA to hg38, with <5% of unmapped reads from all cell lines tested (FIG. 2H). We then examined the reads that aligned to hg38 exactly once (aligned=1, uniquely mapped reads, UMR) and more than once (aligned>1, multi-mapped reads, MMR). UMR were defined by a MAPQ>40 Phred quality score in which the probability of erroneous mapping was <0.0001. Subtracting the number of UMR from the total number of mapped reads returned the number of MMR. Noticeably, nscDNA reads from all cell lines except Colo320DM were dominantly MMR (FIG. 2A), whereas at least 75% of gDNA reads were UMR. Colo320DM nscDNA was an outlier in its higher composition of UMR (59.3+3.9%). There are several high coverage, uniquely mappable areas in this cell line, including the DM locus (FIG. 1E), which would contribute to the higher coverage of UMR.

Since the majority of nscDNA reads were multi-mapped, we questioned whether repetitive elements (short interspersed nuclear element (SINE)/long interspersed nuclear element (LINE)/Simple/Satellite) were major constituents. Interestingly, the dominance of MMR did not correlate with a greater composition of repetitive elements in nscDNA (FIG. 2B, FIG. 2I). Another major source of MMR are nearly identical, recently duplicated sequences, or segmental duplications (SDs, also called low copy repeats). SDs are DNA segments (>1 kb) that occur more than once in the human genome and share a very high sequence identity (>90%) to each other (33). Initially, we found multiple high coverage peaks throughout the genome corresponding to SDs in nscDNA (FIG. 2C). We note this observation because ˜13 kb of mtDNA segments are nearly identical with SDs in chr1, chr3, chr5, chr11, and chr14 (FIG. 2J).

We utilized a defined set of SD tracks to quantify the percentage of SDs in nscDNA and gDNA reads, with analyses including (FIG. 2K) or excluding (FIG. 2D) the SD tracks associated with mtDNA. Although the exclusion would most likely underestimate the SD fraction in nscDNA, SD enrichment was still observed in nscDNA. GM12878 and HeLa S3 carried the highest percentage of nscDNA reads attributed to SDs at 12.6+2.0% and 13.2+1.6%, which was more than twice the amount of SDs in corresponding gDNA samples (5.5% and 5.8%, respectively).

We used Southern blotting to directly estimate the sizes of SD-containing nscDNA using a mix of chromosomal DNA-specific (not shared with mtDNA) SD probes (FIG. 2E). 92% of HeLa S3 nscDNA reads were mapped to chrM and only 8% were mapped to DNA of nuclear origins (FIG. 1B). Therefore, a very small fraction of nscDNA would hybridize to the SD-specific probes. Nonetheless, SD signals were evident in nscDNA fragments and sizes ranged from 0.5 kb to over several kb after 45 seconds of sonication.

We further confirmed that SD-containing nscDNA were indeed extrachromosomal by metaphase FISH on HeLa S3 and GM12878 cells (FIG. 2F, FIG. 2L) using two SD-harboring fosmid clones (G248P86779H3/G248P81785G8) as probes. To exclude false-positive signals, we applied a very stringent criteria and labeled the probes with either DIG (red) or Biotin (green) and searched for signals in which co-localization (yellow) occurred. We observed signal co-localization in multiple chromosomes as well as outside of chromosomes. In summary, 16 (G248P86779H3) and 20 (G248P81785G8) out of 25 metaphases in HeLa S3 cells exhibited extrachromosomal signals (FIG. 2G), while 15 (G248P86779H3) and 19 (G248P81785G8) out of 25 showed signals in GM12878 (FIG. 2M). Many of the metaphases had multiple extrachromosomal signals. In contrast, extrachromosomal signals were very rare in metaphases hybridized to a BAC clone (RP11-685G23) from a single-copy genomic region that was not enriched with eccDNA. Despite covering a much larger genomic region (163 kb) than fosmid clones (<40 kb), only three metaphases showed extrachromosomal signals with RP11-685G23.

Dominance of Duplicated Sequences in Human and Mouse Sperm nscDNA.

Since SDs are known to contribute disproportionately to copy number variations within humans and between primates, we hypothesized that nscDNA may act as mediators in these events given their high SD composition in cell lines. We approached this question by enriching for nscDNA from pooled frozen human semen (0.5 mL/replicate) and mature mouse sperm extracted from the epididymis of four C57BL/6 mice (two mice/replicate). Because mtDNA could possibly be degraded technically during semen preservation or biologically during the process of eliminating paternal mtDNA, we developed an exogenous plasmid-based quality control method to quantify the enrichment of nscDNA by spiking circular pUC18 and linearized pEGFP-C1 (double digested) into lysed sperm cells. Following enrichment, we obtained circular:linear plasmid ratios of 738+149 and 1,418+254 for human and mouse nscDNA, respectively (FIG. 3A). Following quality control, we directly examined nscDNA from human sperm using transmission electron microscopy (TEM) (FIG. 3I). Unlike high molecular weight gDNA, we observed a great degree of negative staining in nscDNA, possibly due to the high density of DNA in large supercoiled structures.

Importantly, our approach enabled us to estimate the absolute amount of nscDNA per sperm cell, which has not been possible in RCA-amplified DNA. We recovered 18-58 ng of nscDNA from mouse sperm after exonuclease treatment, which amounts to 0.9-6.5 fg of nscDNA/cell (Table 1). The number of mouse sperm and the amount of nscDNA remaining after enrichment (post-exonuclease reaction) was used to quantitate the amount of nscDNA per sperm cell. Since a single mouse haploid nucleus contains 3 μg of DNA, we could infer that nscDNA accounts for up to 0.2% of nuclear DNA in sperm. This figure could be underestimated given that the loss of nscDNA at each purification step was not considered.

TABLE 1
The stepwise enrichment and quantification of nscDNA in mouse sperm samples.

| | | | DNA | | DNA | Total | nscDNA |
|---|---|---|---|---|---|---|---|
| Mouse | Number | Pre- | input | Post- | input | nscDNA | amount/ |
| sperm | of | exonuclease | amount | exonuclease | correction | amount | cell |
| samples | sperm | (ng) | (ng) | (ng) | factor | (ng) | (fg) |
| nscDNA-1 | 21,000,000 | 304 | 290 | 18.3 | 0.95 | 19.2 | 0.9 |
| nscDNA-2 | 9,500,000 | 745 | 700 | 57.9 | 0.94 | 61.6 | 6.5 |

We sequenced human and mouse sperm nscDNA and mtDNA was not enriched (<0.2% in human and mouse). Instead, we found that nscDNA reads were dominantly MMR (human: 82.2+1.4%, mouse: 96.6+0.9%) (FIG. 3B). We noticed that, unlike cell line-derived nscDNA, sperm nscDNA carried greater total repetitive elements than gDNA, due to the profound dominance of satellite DNA in both human (52.9+1.7%) and mouse (81.6+0.6%) samples (FIG. 3C). Significantly, human nscDNA was four times more enriched with SDs than gDNA (nscDNA: 21.2+1.1%, gDNA: 5.7%) (FIG. 3D). The presence of SDs in human sperm nscDNA was also confirmed through Southern blotting (FIG. 3J). Consistent with the PFGE for HeLa S3 nscDNA (FIG. 1C), sperm nscDNA showed very different mobility in responses to sonication. Genomic DNA was readily sonicated and became smaller in length, whereas nscDNA was trapped in wells with little changes by sonication. In corroboration with the NGS data (FIG. 3C), both SDs and satellite DNA were enriched in nscDNA relative to genomic DNA.

SD enrichments were exemplified by high coverage peaks of nscDNA in the genomic regions with multiple SDs (FIG. 3E). Some of the high coverage regions overlapped between SDs and satellite DNA (FIG. 3K) or were only attributed to satellites (FIG. 3L). Given that (1) SD tracks are not repeat-masked, and (2) SDs overlap with satellite DNA in pericentromeric regions, we cautioned that the overrepresentation of SDs in nscDNA would be as a result of the dominance of satellite DNA. After removing the intersection between SDs and satellite DNA, however, we found that SDs remained overrepresented in human sperm nscDNA (13.9+0.1%) (FIG. 3F). With the dominance of SDs and satellite DNA, UMR remained a minor fraction (FIG. 3B). Reads mapped to SDs were more abundant than total UMR in both human (3.4 times) and mouse (5.9 times). The ratio of SD-mapped reads to UMR was 42-fold and 67-fold higher in nscDNA than in gDNA in human and mouse, respectively (FIG. 3G).

These results indicate that circular DNA molecules in sperm, small or large, would arise predominantly from duplicated regions of the genome. This notion was further validated for human nscDNA by mapping to the most recent, telomere-to-telomere assembly of the haploid CHM13 cell line genome. This assembly fills many of the gaps in hg38, which significantly coincide with SDs. The mappability of sperm nscDNA to hg38 was low; on average, only 62.0+4.8% of reads were mapped to the hg38 genome (FIG. 3H). With the CHM13 genome, we observed a dramatic increase (23.4%) in the mappability of human sperm nscDNA, while gDNA mappability remained almost the same between hg38 (96.1%) and CHM13 (96.9%). Thus, the nearly identical SDs and other sequences in hg38 gaps are the significant source of nscDNA.

Fusion Junctions of nscDNA.

Highly identical sequences could promote the formation of nscDNA by homology-directed rearrangements. Understanding the mechanism of nscDNA formation requires precise, nucleotide-level information at fusion points. Previous studies of eccDNA have relied on the analysis of discordantly aligned read pairs with outward orientation (in the opposite direction) as evidence of circularization. Examples of this were observed in the TWIST2 gene (chr2) and NSUN6 gene (chr10) in human sperm nscDNA, which contained peaks with outwardly-oriented read pairs (red bars), indicating hotspots for nscDNA formation (FIG. 4A). In mouse sperm nscDNA, paired reads of outward orientation encompassed part of the ASMT gene in chrX (FIG. 4D).

To confidently identify specific sequences contributing to nscDNA fusion points, UMRs are necessary. To do so, we turned to Colo320DM nscDNA data and investigated fusion points in 8q24.1, a locus surrounding theMYC gene that contains very few SDs. The deep coverage of nscDNA in the amplified locus in Colo320DM (FIG. 1E) facilitated our survey for fusion points involved in circular DNA formation. In most of the region, the coverage ratio between nscDNA and gDNA was between 0.1 to 0.2 (FIG. 4B). We noticed that, in the 6 kb region within the long non-coding enhancer RNA (lncRNA) PVT1, the nscDNA:gDNA coverage ratio was above 0.2, which may indicate a hotspot for the origin of small-sized nscDNA.

We extracted the split reads from the PVT1 region for further analysis. We assumed that a single split read where both halves map to the same strand, harbors a single fusion point of circular DNA, and the distance between the coordinates of each end provides the size of the nscDNA (FIG. 4C). Nucleotide-level analysis at these fusion points would provide information on how two ends fuse. 52% of fusions were mediated by 1-10 bp of microhomology, whereas 40% of fusion events lacked homology. Importantly, there were three events with more than 50 bp segments (82 bp, 114 bp, and 140 bp) exhibiting 77-93% homologies. Thus, nscDNA formed using long homology, indicating that duplicated sequences could promote the formation of nscDNA. Since the majority of nscDNA stemming from the surveyed region were less than 3 kb, they region may be a hotspot for small-sized nscDNA formation.

Other studies laying the groundwork for eccDNA enrichment have relied on exonucleases for linear DNA depletion and RCA for the amplification of circular DNA, with a few exceptions. eccDNA characterization and classification has been largely based on NGS data analyses of eccDNA, which were focused on investigating uniquely mapped regions of the genome. We developed our method of nscDNA enrichment without RCA to reduce potential limitations caused by in vitro amplification (FIG. 1H, 1I). The purity of our preparations was validated by quality controls (mtDNA/spiked plasmids) and PFGE, which provided us a unique opportunity to uncover the characteristics of eccDNA in the naïve state (nscDNA) in a cell. Importantly, our approach allowed us to quantify the absolute amount of nscDNA in a cell (sperm). By molecular analysis, we estimate that the nscDNA populations we obtained range up to tens of kilobases, the size of which the biological significance has been unclear.

The most notable finding of our study is the overrepresentation of segmental duplications (SDs) in nscDNA, the segments in the human genome that are strongly associated with copy number variations. In human and mouse sperm, the overrepresentation was 4-fold and 3-fold, respectively (human nscDNA: 21.2+1.1% and gDNA: 5.7%, mouse nscDNA:17.6+1.8% and gDNA: 5.6+0.3%) (FIG. 3D). This enrichment was not a mapping artifact of the enrichment of satellite DNA, which overlaps with SDs in pericentromeric regions. Even after accounting for overlaps with satellite DNA, there still be a 3-fold enrichment of SDs in nscDNA (human nscDNA: 13.9+0.1% and gDNA: 4.8%) (FIG. 3F). With the overrepresentation of these nearly identical SDs and satellite DNA, uniquely mapped reads (UMR) were depleted. With such dominance of multi-mapped reads (MMR), it is difficult to explicitly define where SD-derived nscDNA originates in the genome based on our current, short sequencing reads-based approaches. A more complete reference genome, such as the CHM13 genome assembly, along with ultra-long-read sequencing technologies, could help to define the origins of nscDNA in these structurally diverse areas of the human genome.

Previous studies describe repetitive DNA elements as a significant component of eccDNA. As defined by RepeatMasker, we found that satellite DNA was the major constituent of both human and mouse sperm nscDNA at 52.9+1.7% and 81.6+0.6%, respectively (FIG. 3C). Satellite DNA accounts for 10% of the human genome, forms the centromeric locus and pericentromeric heterochromatin. Satellite DNA is also observed in eccDNA from plants, fruit flies, mice, and humans. Most of these findings were obtained by two-dimensional (2D) agarose gel electrophoresis, a molecular technique that directly characterizes eccDNA based on size and topology by incorporating labeled probes to identify specific genomic elements. Our studies confirmed these observations by genomic technologies.

In contrast to satellite DNA, SINEs and LINEs were depleted in nscDNA reads from both human (SINEs: 7.1+0.3%, LINEs: 1.5+0.04%) and mouse sperm (SINEs: 0.4+0.05%, LINEs: 0.9+0.2%) (FIG. 3C). SINEs and LINEs are groups of transposable elements (TEs) making up 34% (SINEs: 13% and LINEs: 21%) of the human genome. Low distributions of Alus, the most common SINE elements, were also noted in the sequencing data of microDNA from human maternal plasma that were not in vitro amplified. C57BL/6 mouse sperm nscDNA composition differs from that of in vitro amplified microDNA, which is preferentially derived from full-length LINE-1 retrotransposons compared to various other tissues. This indicates that repetitive DNA composition may differ between eccDNA size populations. The depletion of these interspersed elements in nscDNA may be due to the depletion of single-copy regions where SINE and LINE elements scatter. We found an exception to this when analyzing nscDNA from Colo320DM (FIG. 1E), which retained high coverage of single-copy genomic regions including 8q24.1, where there are few duplicated segments, but many repetitive elements. This indicates that eccDNA may arise from different mechanisms in cancer cells versus normal cells. In cancer cells, large eccDNA species may be generated as a result of genome instability throughout the genome.

nscDNA populations from normal human lung and mouse liver tissues were also found to be dominant with SDs (FIG. 4E-FJ). The presence of nscDNA with similar features in several organs hints that nscDNA likely arises from a general cellular process, such as DNA replication in normal cells. The majority of SDs in humans initially formed ˜35 to 40 million years ago during the surge of a younger Alu family of transposable elements, and once formed, SDs became the core of additional rearrangements, leading to regions of complex genome architecture. Complex architecture could promote the formation of nscDNA during replication when replication forks stall and break due to tandemly aligned SDs (and satellite DNA) forming secondary DNA structures, and strand invasion of broken DNA would initiate the formation of circular DNA (FIG. 4K). Complex genomic regions can be fragile and subject to spontaneous breaks.

The fate of SD-bearing nscDNA (formation, re-integration and degradation) in germ cells could be associated with genetic variations, since SDs are subject to meiosis-specific DNA double-strand breaks, which would facilitate the integration of nscDNA into chromosomes. A trace of this mechanism can be found within SD clusters of the human genome. In yeast and bovine, circular intermediates have been implicated in chromosomal amplifications or genic translocation events. It will be profound if future research can touch on the possibility of the involvement of circular DNA in genome evolution.

### Materials and Techniques

Purification and Enrichment of nscDNA.

The Plasmid Midi Kit (Qiagen, 12143) was used to purify circular DNA from cell lines (80×10⁶-100×10⁶ cells), sperm, and tissue samples. Human sperm nscDNA was extracted from 1 mL of pooled semen (Innovative Research, IR100076), which was split into two technical replicates (450 μL/nscDNA replicate, 50 μL for gDNA). Mature mouse sperm was isolated from the epididymis of four C57BL/6 mice (2 mice/biological replicate) that were sacrificed at 35 weeks and 33 weeks old, respectively. 21×10⁶ and 9.5×10⁶ total sperm were collected from each replicate. gDNA was extracted from 1×10⁶ mouse sperm. Liver tissue (370 mg total) from the second pair of mice was extracted and combined before homogenization. A fresh, normal lung tissue sample (frozen) was obtained from the Cedars-Sinai Cancer Biobank and Translational Research Core under protocol Pro00052428 (Circular small DNA as a new cancer biomarker). nscDNA from 50 mg of human lung tissue was extracted twice (technical replicates). nscDNA extraction from sperm and tissues required the addition of Proteinase K during cell lysis (500 μg/mL), followed by incubation at 55° C. for 1.5 hours (overnight for lung tissue). Sample eluates were phenol-chloroform extracted before DNA precipitation.

The removal of residual, linear double-stranded chromosomal DNA was facilitated by Plasmid-Safe ATP-Dependent DNase (Epicentre, E3101K) digestion reactions. Up to 750 ng of purified nscDNA (pre-exonuclease) was digested in 300 μL reactions containing 60 U enzyme and 1 mM ATP and 1× reaction buffer. The reactions were incubated at 37° C. for at least 16 hours, followed by enzyme inactivation by incubation at 70° C. for 30 minutes. nscDNA was further purified using DNA Fast Flow PCR Grade centrifugal filters (Microcon, MRCFOR100ET) to exchange buffers and concentrate nscDNA for downstream applications. nscDNA concentration was measured before and after digestion with a Qubit 3.0 fluorometer using the dsDNA HS Assay Kit (Life Technologies, Q32851).

nscDNA and gDNA Library Construction and Sequencing.

DNA was fragmented by either Bioruptor Standard (Diagenode) or Covaris M220 sonicators to −350 bp length. Libraries were created with the NEBNext Ultra II DNA Library Prep Kit for Illumina (NEB, E7645), starting with 10 ng of DNA input from cell line samples. 2-5 ng of input DNA was used for sperm and tissue samples. The protocol was followed per manufacturer's instructions, bypassing the DNA size selection step. 7 PCR cycles were used for the PCR amplification step.

The sequencing reads were trimmed using Trim Galore (v.0.6.1) and Cut Adapt (v2.3) to remove adapters and subsequently aligned to the UCSC hg38 human reference genome or mm10 mouse reference genome using Bowtie 2 (v2.3.5). Reads were mapped with unpaired alignment unless otherwise indicated. To filter for uniquely mapped reads, only reads with mapping quality scores=>40 were included. Read depth was normalized using a per-million scaling factor from the total number of mapped reads after filtering. Browser extensible data format (BED) files were generated using Bedtools (v2.28.0) and mapped into 1 kb non-overlapping bins. Coverage maps were visualized using the Interactive Genome Viewer (IGV) (v2.5.0) (61). The hg38 and mm10 reference genomes were downloaded from the UCSC Genome Browser at hgdownload.soe.ucsc.edu/goldenPath/hg38/bigZips/and hgdownload.soe.ucsc.edu/goldenPath/mm10/bigZips/, respectively. The CHM13 reference genome was provided by the Eichler lab. The analyzed datasets were uploaded to the Sequence Read Archive (SRA) database with BioProject ID PRJNA641068 (human) and PRJNA655921 (mouse).

### Identification of Segmental Duplications and Repetitive Elements.

To gauge the segmental duplication and repetitive DNA contents, repetitive elements in RepeatMasker and segmental duplications (genomicSuperDups) from the UCSC Genome Browser (hgdownload.soe.ucsc.edu/goldenPath/hg38/database/for hg38 and (hgdownload.soe.ucsc.edu/goldenPath/mm10/database/for mm10) were intersected with the gDNA and nscDNA sequencing reads. CHM13 SD tracks were provided by the Eichler lab. Reads mapped in 1 kb non-overlapping bins acted as the control for the repetitive element and segmental duplication tracks. For repetitive element analysis, only reads mapping to SINEs, LINEs, Satellites, and simple repeats were considered. Due to the enrichment of circular mtDNA during nscDNA isolation, segmental duplications on nuclear chromosomes with high similarity to those on chrM were excluded from the cell line analysis.

### Example 2. Exemplary eccDNA Analysis Kit

### Extraction Component Step

Cell/tissue lysis buffer
Suspension buffer
Neutralization buffer
Plasmid prep column
Column wash buffer
Elution buffer

### Purification Component Step

Exonuclease
   Examples: ExoV linear DNA digestion enzyme plus buffer, PLASMID-SAFE™ ATP-Dependent DNase plus buffer
Concentration column and elution buffer

Quantification and/or validation component step—determine successful isolation of eccDNA (e.g., by qPCR)—new addition or improvement to the isolation kits or isolation methods of eccDNA.
(Circular) DNA standards—4 plasmids from 5 kb to 150 kb Standards:
1. pEGFP-C1 (4,731 bp, linearized by double-digestion with BamHI and EcoRI)
2. pCMV-Cre (6,240 bp, circular plasmid)
3. WI2-2061P6 (46,225 bp, circular Fosmid)
4. RP11-615L21 (155,670 bp, circular BAC Clone) q PCR primer mixture for the standards

TABLE 2
Primer sequences for qPCR analysis of different controls/standards.

| | | | SEQ | | SEQ |
|---|---|---|---|---|---|
| | | | ID | | ID |
| Control type | Control name | Forward Primer | NO: | Reverse Primer | NO: |
| circular | Chromosome M | 5′- | 1 | 5′- | 2 |
| mtDNA | | TATTTCGCCCACTAAG | | CGGATGCTACTTGTCC | |
| (endogenous | | CCAATC-3′ | | AATGATGG-3′ | |
| control) | | | | | |
| linear dsDNA | Chromosome 17 | 5′- | 3 | 5′- | 4 |
| (endogenous | | GCAATCTGTTAGTAG | | GCAGGAACTGAAAGA | |
| control) | | GCAGTGGTGG-3′ | | AGTGGGTG-3′ | |
| Linear | pEGFP-C1 | 5′- | 5 | 5′- | 6 |
| Plasmid | | CACATGAAGCAGCAC | | TCCTTGAAGTCGATGC | |
| (exogenous | | GACTT-3′ | | CCTT-3′ | |
| control) | | | | | |
| Circular | pCMV-Cre | 5′GAACGAAAACGCTG | 7 | 5′- | 8 |
| Plasmid | | GTTAGC-3′ | | CCCGGCAAAACAGGT | |
| (exogenous | | | | AGTTA-3′ | |
| control) | | | | | |
| Circular | WI2-2061P6 | 5′- | 9 | 5′- | 10 |
| Fosmid Linear | | CGTTTCCGTTCTTCTT | | TGTTCATTCCACGGAC | |
| (exogenous | | CGTC-3′ | | AAAA-3′ | |
| control) | | | | | |
| Circular BAC | RP11-615L21 | 5′- | 11 | 5′- | 12 |
| Clone | | CAACTCAATCGACAG | | GGCTTTGTTTGCCGTA | |
| (exogenous | | CTGGA-3′ | | ATGT-3′ | |
| control) | | | | | |

### Example 3. Exemplary nscDNA Isolation Methods and Results

FIG. 5 shows an overview of the nscDNA isolation technique in Example 1.

FIG. 6A shows the overview of another nscDNA isolation technique. FIG. 6B shows the ‘control’ circular DNA molecules for the nscDNA isolation technique. Compared with the technique in FIG. 5, herein the alkaline buffer P2 is used after proteinase K and RNase A, because proteinase K and RNase are more active in lysis buffer than in the alkaline buffer P2.

For qPCR analysis, first, the relative quantity (RQ) of each target is calculated by the following equation, with normalization to genomic DNA:

$$RQ ( circular DNA ) = {2}^{( - Cq ( nscDNA ) - Cq ( gDNA ) )} , \\ RQ ( linear DNA ) = {2}^{( - Cq ( nscDNA ) - Cq ( gDNA ) )} ,$$

where circular DNA can be pCMV-Cre, Fosmid, BAC Clone, and/or mtDNA; linear DNA can be pEGFP-C1; and Cq refers to the quantification cycle values in qPCR (e.g., machine CFX96) that indicates the minimum number of amplification cycles necessary for the fluorescence value of each sample to overcome background fluorescence.

The enrichment of circular DNA over linear DNA is expressed as a ratio, i.e., the RQ (circular DNA) divided by the RQ (linear DNA).

FIG. 7A shows the qPCR results of two human semen samples isolated with the technique shown in FIG. 6A for nscDNA. Plasmids were mixed with Human semen before circular DNA extraction. Human Semen-1 and Human Semen-2 are technical replicates. pCMV primer set targets Cre sequence; Fosmid primer set targets backbone (non-chromosomal); and BAC Clone primer set targets backbone (non-chromosomal).

FIG. 7B shows the qPCR results of three independent breast tumor tissues. Plasmids were mixed with human breast tissue before circular DNA extraction. Some targets weren't analyzed by qPCR due to low amount of nscDNA available. pCMV primer set targets Cre sequence; Fosmid primer set targets backbone (non-chromosomal); and BAC Clone primer set targets backbone (non-chromosomal).

### Example 4

Structural aberrations of chromosomes are the manifestation of tumor cells. Among the aberrantly structured DNA molecules, megabase-sized, circular chromosomes (double minute chromosomes, DMs) have established roles in cancer biology and patient care, as DMs impact cancer progression, tumor heterogeneity, and therapy efficacy. In addition to DMs, smaller eccDNA species also exist in cancer cells. These eccDNA has recently been characterized using in vitro amplification (rolling circle amplification, RCA) followed by next-generation sequencing (NGS). However, two biases introduced by these approaches prevent us from a comprehensive understanding of the eccDNA at the native state. RCA preferentially amplifies smaller eccDNA and eliminates information on epigenetic modifications. Furthermore, NGS data analysis has been limited to uniquely mappable reads. Therefore, the native compositions of eccDNA and the roles in cancer biology remain unclear.

Extrachromosomal circular DNA (eccDNA) carries extra copies of genetic materials and contributes to genomic amplification in tumor cells. eccDNAs lack centromeres and segregate unevenly into daughter nuclei, creating copy number heterogeneity in a cell population. Such heterogeneity provides adaptive potential and significantly impacts cancer progression and therapy efficacy, as manifested by the megabase-sized eccDNA (double minute chromosomes, DMs) carrying oncogenes and therapy target genes. Recent studies have revealed smaller eccDNA species. However, their roles in cancer biology and mechanisms of biogenesis remain elusive.

This ambiguity stems from the lack of quantitative assessment of eccDNA in tumors. Most studies employ sequencing to examine eccDNA that has undergone in vitro amplification, which results in a skewed composition of eccDNA. Furthermore, analyses have focused on the uniquely mappable genome and have excluded sequences from nearly identical, multi-mappable regions.

Herein, we successfully enriched the native composition of eccDNA from tissues without in vitro amplification (see Examples 1-3). Stringent quality control processes showed approximately 1000-fold enrichments of both internal and exogenous circular DNA controls over linear DNA in our approach. Molecular approaches revealed native eccDNA of various sizes, from a few kb to several hundred kb with CpG methylation. Unbiased NGS analysis revealed that segmental duplications (SDs) and satellite repeats are the predominant components of tissue-derived eccDNA in both humans and mice.

Therefore, we have developed a method to efficiently enrich eccDNA of a few kb to several hundred kb in human tissues without in vitro amplification. Our approach systematically interrogates the genomic origins of eccDNA at their native composition in cancer tissues and reveals the real impact of eccDNA in cancer biology. We introduced stringent quality control steps and showed that both internal and exogenous circular DNA controls were enriched more than 1000-fold. Our approach can efficiently isolate eccDNA from 50 mg of human tissues. We will isolate eccDNA from 30 breast tumor tissues and 30 colon tumor/normal adjacent tissues. We will sequence the eccDNA and systematically investigate their genomic origins. We will determine whether small eccDNA also arises from oncogene loci. As epigenetic modifications of eccDNA remain unaddressed, we will examine the differential DNA methylation between chromosomal DNA and eccDNA.

Specific methods include (but are not limited to):

### Tumor Adjacent Normal Pairs

Tumor and normal adjacent tissues will be obtained from the Biobank. 30 breast tumor tissues and 30 colon tumor/adjacent normal tissues pairs will be used for this study.

eccDNA Extraction

Plasmid Midi Kit (Qiagen) will be used for isolating eccDNA from tissues. 50 mg of tissue will be homogenized in the lysis buffer. We will add proteinase K and incubate the lysate at 55° C. for overnight before proceeding to column-based purification. pUC18 and linearized pEGFP-C1 will be added to the cell lysis solutions. After washing, DNA will be eluted and cleaned by phenol-chloroform extraction.

The removal of residual, linear DNA will be done by Plasmid-Safe ATP-Dependent DNase (Epicentre) at 37° C. for at least 16 hours. Following enzyme inactivation at 70° C., eccDNA will be further purified using DNA Fast Flow PCR Grade centrifugal filters (Microcon). The typical yield of eccDNA is 20˜50 ng.

eccDNA Quality Control

As a quality control, we will perform qPCR to measure the relative quantity of pUC18 and linearized pEGFP-C1 in tissue samples. Standard curves (0.01, 0.1, 1, and 10 ng or 0.001, 0.01, 1, and 1 ng) will be created using genomic DNA extracted from the same tissue. The relative quantity (RQ) of targets will be normalized to gDNA and calculated using the following formula: RQ=2\hat{ }(-Cq(nscDNA)-Cq(gDNA)). Ratios will be calculated by dividing the RQ of the pUC18 by the RQ of the pEGFP-C1. Data will be reported as ratio mean+SEM. We will confirm a circular/linear ratio of around 1000.

Eccdna and gDNA Library Construction and Sequencing

Sequencing libraries will be created from 10 ng of eccDNA using the NEBNext Ultra II FS DNA Library Prep Kit for Illumina (NEB). Seven PCR cycles will be used to introduce for introducing index primers into the library. Approximately 100 million, 150 bp paired-end reads will be obtained.

The sequencing reads will be trimmed using Trim Galore (v.0.6.1) and Cut Adapt (v2.3) to remove adapters and subsequently aligned to the UCSC hg38 human reference genome using Bowtie 2 (v2.3.5). The output reads will be mapped to hg38, and the reads will be counted in non-overlapping 1 kb bins throughout the entire genome. Each of the approximately 3 million bins will be assigned a value based on the number of reads mapping in the bin. The number of reads within each bin will be divided by a per-million scaling factor (for example, the scaling factor is 100 for a run with 100 million reads) in order to adjust for the total sequencing depth of a particular sequencing run (Normalized read coverage, NRC). Browser extensible data format (BED) files will be generated using Bedtools (v2.28.0) and mapped into 1 kb non-overlapping bins. Coverage maps will be visualized using the Interactive Genome Viewer (IGV) (v2.5.0).

### Duplications, Repetitive DNA and Uniquely Mapped Reads

Repetitive elements (SINEs, LINEs, satellites, and simple repeats) in RepeatMasker and SDs (genomicSuperDups) from the UCSC Genome Browser will be intersected with the eccDNA sequencing reads. To filter for UMRs only reads with mapping quality scores≥40 will be included.

### Validation of Circularity

To validate the circularity of eccDNA, we will employ an approach in which eccDNAs was detected by at least two overlapping structural-read variants (discordant paired-end reads and split reads with both start and end coordinates of putative eccDNA junctions). Split reads will be obtained by remapping nonmapped fragments of soft-clipped reads using BWA-MEM. Based on the split reads, we will design specific primer sets and amplify fusion points by PCR. We will also examine whether highly similar sequences are located at the fusion points of eccDNA.

eccDNA in Breast and Colon Tumors

Our methods of enriching eccDNA can cover eccDNA of a broad size range (a few kb to several 100 kb), the range that hasn't been extensively studied before. General characterization, such as repetitive DNA contents and duplicated segments, will determine whether satellite DNA and SDs are also dominant genomic elements in eccDNA. We will then identify eccDNA by read-depth analysis in uniquely mappable areas of the genome. Uniquely mappable genome covers 80% of the genome and most of the cancer-related genes. So, UMG analysis should provide deep insights into the role of eccDNA in cancer biology. We will rigorously investigate whether eccDNA harbors any cancer genes and falls into recurrently amplified regions in TCGA data. When eccDNA covers genes with unknown significance in cancer, we will find cell lines in the CCLE database that have focal amplification in the region covering the eccDNA. We will examine that focal amplification is the result of eccDNA by FISH. We will then knock down genes within eccDNA and examine cell growth.

As a preliminary analysis, we show here NRC of the 11 Mb region, harboring CCND1 oncogene, for breast tumor genomic DNA (gDNA) and eccDNA. In tumor A, this region is amplified and is associated with the enrichment of eccDNA.

Breast tumors are the most common female cancer and 1 in 8 women suffers from breast tumors in their lifetime. One issue is that adjacent normal breast tissues are, in most cases, not available. For colon tumors, we will have tissues from normal adjacent tissues. We will process normal tissues for eccDNA and determine whether eccDNA seen in tumors is cancer-specific and not present in paired normal tissues.

Association with Genomic Amplification

For tumors with oncogene eccDNA and multiple eccDNA in the genome, we will investigate whether eccDNA loci are amplified. The analysis will be done by Whole Genome Sequencing (WGS). We anticipate that eccDNA derives from one of the alleles. We will determine the allele-specific formation of eccDNA by Patchwork.

CpG Methylation of eccDNA

One of the advantages of eccDNA isolation without in vitro amplification is to preserve DNA methylation information, as in vitro amplification cannot copy methylated cytosines. It is currently unknown whether eccDNA mirrors the methylation patterns of native genomic loci or whether eccDNA has unique methylation profiles, for example, hypomethylation.

Here we show that eccDNA isolated by our approach retains CpG methylation. We used an ELISA-based assay for the quantification of global DNA methylation (Global DNA Methylation-LINE-1 Kit, Active Motif), and found the varying degree of eccDNA methylation between normal lymphoblastoid cells (GM12878) and Hela. As expected, CpG methylation became almost undetectable after in vitro amplification (RCA).

The most comprehensive way of profiling DNA methylation genome-wide is whole-genome bisulfite sequencing. However, this technique requires roughly one μg DNA. A small amount of DNA is feasible for a microarray-based approach (Infinim Methylation EPIC Beads Chip, Illumina). The Chip is designed to study methylation in important genomic segments throughout the genome, but may not comprehensively cover the region where eccDNA derives. In such a case, we will employ classical, PCR-based targeted bisulfite sequencing combined with next-generation sequencing.

### Example 5—Method of Isolating eccDNA Utilizing Agarose

### DNA Extraction

For preparation of agarose plugs, nuclei from cells or tissues will be washed twice with PBS, resuspended in 45 ul PBS, and incubated at 43° C. for 2 min.

Control plasmids will be added into the resuspended nuclei.

One percent low melting agarose (in 100 mM EDTA) will be melted at 90° C. followed by incubation at 45° C. for 10 min. Melted agarose (45 ul) will be added to the resuspended cells at a final concentration of 0.5% and mixed gently. The mixture will be immediately cast into a plug mold and plugs were incubated at 4° C. until solidified.

Plugs will be incubated at 37° C. for 2 days with freshly added 10 ul Proteinase K (20 mg/ml stock concentration) in 250 ul lysis buffer (50 mM Tris pH 8.0, 100 mM EDTA, 0.2% SDS) for each 90 ul plug, with occasional shaking.

Plugs will be washed three times by adding 1 ml (per plug) TE buffer (10 mM Tris, pH 8, 1 mM EDTA), manually shaking for 10 s and discarding the wash buffer before adding the next wash.

Next, plugs will be incubated with 5 ul RNaseA (10 mg/ml) in 250 ul ml (per plug) TE buffer for 1 h at 37-C with occasional shaking. Plugs will be then washed four times by adding 1 ml (per plug) wash buffer (10 mM Tris, pH 8, 50 mM EDTA), and shaking for 15 min on a horizontal platform mixer at 180 rpm at room temperature. For the final washing, we will add 1×Protease inhibitor cocktail (Sigma P8340) to inactivate Proteinase K with AEBSF.

Following washes, plugs will be stored at 4-C in wash buffer or used for exonuclease digestion.

### Exonuclease Digestion

Plugs will be washed 4 times with 5 ml of 10 mM Tris-HCl, pH 8 followed by a single wash (1 hr) with 500 ul exonuclease reaction buffer (NEBuffer 4). Each plug (with Xg genomic DNA) is digested with exonuclease at 37-C for about 16 h.

For recovery of DNA from the gel, agarose will be washed 3 times in 10 ml TE buffer (10 mM Tris, 1 mM EDTA, pH 8) and melted at 70-C for 5 min followed by incubation at 43° C. for 10 min. Next, 2 ul agarase (0.5 U/ul, 1 U/100 mg=100 ul agarose, Thermo Scientific) will be added to each tube for digestion of the agarose and incubated for 1 h at 43-C. The DNA was purified from agarose by isopropanol precipitation.

### Quality Control Steps

Enrichment of circular DNA is evaluated by quality control qPCR.

### Example 6

Methods and Quality Control Steps for Enriching Very Large eccDNA:

The above examples demonstrated the successful enrichment of mtDNA (16 kb). We had not included controls larger than mtDNA, and the enrichment of 50-100 kb eccDNA was not evaluated. It is unknown whether the approach would efficiently enrich very large eccDNA. To address this item, we included Fosmid (WI2-2061P6, 46 kb) and BAC (RP 11-22012, 102 kb and RP11-118I10, 104 kb) clones as exogenous control circular DNA (FIG. 8A). The three exogenous circular DNA, modified pCMVCre (mCre, 4.5 kb), Fosmid and BAC clones would mimic eccDNA of a wide size range. Additionally, we digested sperm proteins with proteinase K in a neutral lysis buffer before denaturing DNA in an alkaline solution. Although Proteinase K works in a wide range of pH (7.5-12), the lysis solution has better pH (8.0) for proteinase K than the alkaline solution.

We spiked human sperm with a mixture of these controls. The copy numbers of spiked clones were normalized to genomic DNA (isolated after spiking with the clones), and the ratios to the linear DNA control (linearized pEGFP-C1, 4.7 kb) were determined by qPCR (FIG. 8B). Primer sets were specific to each clone: primers for EGFP in pEGFPC1, primers for Cre in mCre, and primers for the backbones of Fosmid and BAC clone. Very high enrichment was noted for all three controls. The 4.5 kb mCre was enriched more than 3000-fold. Fosmid and BAC controls were also enriched more than 100-fold.

Using ILLUMINA sequencing, we found that the eccDNA isolated in this approach was enriched for segmental duplications (>20%) and satellite DNA (>50%).

Small total eccDNA yields was encountered. Described in the examples above, the final step of eccDNA recovery after ExoV treatment was the recovery of DNA by size-exclusion columns. Traditional, phenol/chloroform-based recovery of eccDNA was found to produce twice as much eccDNA yields as the column-based approach (data not shown). We further tested the enrichment of a BAC clone (RP11-118I10, 104 kb) from these two methods and found the enrichment to be efficient (>100-fold) and comparable between these two methods (FIG. 8B).

Long read, single molecule sequencing of eccDNA in human sperm: We proposed single-molecule sequencing for eccDNA by ONT long-read sequencing. While not wishing to be bound by any particular theory, we believe that the tagmentation-based sequencing library would allow us to sequence full-length eccDNA molecules, revealing the entire DNA contents of large eccDNA molecules without the need for reconstruction of each molecule. We report here that we were able to sequence large (17.5-32 kb) eccDNA from human sperm. These molecules included both simple circular molecules of mono-locus origins and, strikingly complex circular molecules consisting of segments from more than one chromosome.

To do this, we enriched eccDNA from a human semen sample spiked with exogenous pmCre (4.6 kb), Fosmid (WI2-2061P6, 46 kb), and BAC (RP11-118I10, 102 kb) clones. The control enrichments were confirmed by qPCR. With 35 ng of purified eccDNA, we created a sequencing library using ONT's RAPID sequencing kit. The distribution of 65,949 reads is shown in FIG. 9A. We confirmed the efficient recoveries of full-length pmCre and mitochondrial DNA, as these DNA showed prominent peaks at expected sizes (FIG. 9B). We also found that the Fosmid-derived sequence formed a peak around 45-46 kb (FIG. 9A).

We expect that reads coming from full-length eccDNA should have (1) neighboring coordinates at the start and end of the reads and (2) reads consisting of at least two genomic segments (at least one breakpoint). Reads coming from uniquely mappable regions of the genome are needed to confirm successful single-molecule sequencing of full-length eccDNA based on these criteria. We extracted all the reads between 17.5 and 32 kb, between mtDNA monomer and dimer, and looked for the genomic origins of reads using Blat. Thirty-six reads were mapped uniquely to chromosomal DNA (hg38) (Table 3). The distance between the start and end coordinates was less than 50 bp in 23 reads (less than 20 bp in 19 reads). Five other reads were nearly full-length, as the distance was 102, 171, 504, 663, 5224 bps. In total, 28 reads (74%) likely represent near full-length eccDNA. Importantly, four reads have more than two segments, with three of them consisting of segments of multiple chromosomes (interchromosomal). In this regard, five other reads could represent part of eccDNA as they consisted of segments from two chromosomes.

TABLE 3
The DNA contents of 85 long reads

| Sequence origins | 85 total |
|---|---|
| mtDNA | 5 |
| Spiked Fosmid clone | 20 |
| Spiked BAC clone | 1 |
| Segmental duplications | 2 |
| Simple repeats | 21 |
| Uniquely mapped | 36 |
| Full-length eccDNA | 28 |
| Intrachromosomal | 25 |
| Interchromosomal | 3 |
| (likely) part of eccDNA | 5 |
| undetermined | 3 |

These results showed that our single-molecule sequencing strategy could sequence full-length eccDNA. Along with spiked controls, the library was enriched with eccDNA (at least 33/36 uniquely mapped reads). Finally, 19.8% of 65,949 reads were mapped to SDs. The overrepresentation is consistent with our published study.

### SUMMARY

We have developed a prototype of an approach for isolating and analyzing a wide size range of eccDNA in the native state. Our technology includes stringent quality control steps, which will serve as powerful tools to develop fully functional approaches. We are able to sequence entire eccDNA molecules on the ONT platform with a single tagmentation-based library, validating the framework of our technology. While only a small amount of template DNA (35 ng) is sufficient for sequencing, (1) optimizations to our protocol would result in reductions in the amount of DNA required, and (2) increased amount of eccDNA for library construction would improve the robustness of ONT sequencing. Indeed, we obtained more than 1.2 million reads aligned to hg38 from the library constructed with 100 ng eccDNA (data not shown).

### Example 7

Establish an Optimal Approach for Isolating and Sequencing Large eccDNA.

We have shown above that eccDNA can be a significant fraction of nuclear DNA (0.2% in mouse sperm). It remains unclear whether we were able to capture the wide range of sizes of eccDNA efficiently. Such a method would be crucial to improve our understanding of the biomedical role of eccDNA. In response to the Focused Technology Research Development FOA, we continue to develop a method for isolation of eccDNA without in vitro amplification.

We develop a method to reveal the basic characteristics of eccDNA comprehensively by nearly an unbiased collection. Nanopore sequencing provides DNA base calling at the single molecule level up to several hundreds of kilobases. DNA base calling includes base modifications such as methyl-cytosine. While not wishing to be bound by any particular theory, we believe that applying single-molecule Nanopore sequencing allows us to sequence the entire eccDNA molecules and broaden our understanding of the size, genomic origins, and epigenetic modifications of eccDNA.

ONT platform allows the sequencing of DNA molecules consisting of more than a million bases. Therefore, we can characterize a wide size range of eccDNA molecules. To do so, we implement a method to introduce a single sequencing adapter to each eccDNA molecule by transposase-based tagmentation (Rapid Sequencing Kit, ONT), which requires only ˜15 minutes of hands-on preparation time. A motor protein is loaded for each adaptor, and each strand passes independently through the nanopore to generate signals from bases until the end of the DNA molecule, providing the size and nucleotide information of an entire molecule. We predetermine candidate tagmentation conditions by PFGE. The Nanopore reads representing the full-length of spiked fosmid and BAC clone controls validates our conditions and approaches. With a predetermined condition, we have demonstrated that single insertion of an adaptor could provide base calling for entire large eccDNA molecules.

Epigenetic information is obtained from endogenous eccDNA using standard methods. We then confirm subsets of the modifications manually by sodium bisulfite modifications, PCR amplification, and Sanger sequencing.

Isolation of eccDNA. Prior to isolation, circular DNA controls (mCre, Fosmid and BAC clones) and a linear DNA control (pEGFPC1) is added to each sample. (We treat circular DNA controls with exoV before adding to each sample to make sure that the spiked molecules are free of linear molecules.)

Human sperm nscDNA is extracted from 1 mL semen (Innovative Research, IR100076). We split the 1 ml semen into two technical replicates, and 50 ml will be saved for genomic DNA extraction (450 μL for nscDNA, 50 μL for gDNA). Mature mouse sperm is isolated from the epididymis of four C57BL/6 mice (two mice/biological replicate), sacrificed around 34 weeks old. Approximately 10-20 million sperm will be collected from each replicate. As samples representing solid tissues, liver tissues will be obtained from the sacrificed mice and homogenized before eccDNA extraction.

A protocol for neutral cell lysis: nscDNA extraction from sperm and tissues begin with the addition of Proteinase K (500 μg/mL) during cell lysis, followed by incubation at 55° C. for 1.5 h in a neutral cell lysis buffer (10 mM Tris-HCl pH 7.5, 25 mM EDTA, 0.5% SDS, 100 mM NaCl). We then add P2 (alkaline) and P3 (neutralization) buffers of the Plasmid Midi Kit (Qiagen, 12143). After precipitating large macromolecules and chromosomal DNA, we take supernatant and capture low molecular weight DNA from with the QIAGEN column. Sample eluates are phenol-chloroform extracted before DNA precipitation.

The removal of residual, linear double-stranded chromosomal DNA is done by Plasmid-Safe ATP-Dependent DNase (Epicentre, E3101K) and Exonuclease V (NEB). Up to 750 ng of crude eccDNA through QIAGEN columns is digested with 60 U enzyme and 1 mM ATP and 1×reaction buffer at 37° C. for at least 16 h. Exonuclease will be inactivated by incubation at 70° C. for 30 min and removed by phenol/chloroform extraction. eccDNA will be precipitated by ethanol. Yields will be determined by a Qubit 3.0 fluorometer.

eccDNA quality control measures. For each preparation, qPCR is used to estimate the enrichment of circular DNA over linear DNA. With a specific primer set for each control, we estimate the copy number in both eccDNA preps and genomic DNA in quadruplicated reactions. Normalized Cq values of eccDNA (Cq values circular controls in eccDNA prep/Cq values of circular DNA controls in genomic DNA) is divided by a normalized Cq value of linear DNA control (linearized pEGFPC1) to generate enrichment values.

We also examine the mobility of eccDNA in PFGE. We run 25 ng of eccDNA and genomic DNA side-by-side. Samples are run on a CHEF Mapper XA System (Bio-Rad) in 0.5×TBE for 8 hours at 14° C. using the auto algorithm function for 1-200 kb. Gels are stained in the dark with 1X SYBRGold (Invitrogen, S11494) and be scanned on a ChemiDoc Imaging System (Bio-Rad).

Sequencing library construction. We use the transposase-based Rapid Sequencing Kit from ONT. This kit prepares the sequencing library by the tagmentation of DNA by the transposase, followed by the ligation of the sequencing adaptor to the tag. While not wishing to be bound by any particular theory, we believe that a single tagmentation in circular DNA would create a linear dsDNA representing the full-length molecule, with sequencing adaptors on both ends. We demonstrated the method for eccDNA molecules from human sperm.

We optimize the tagmentation process so that eccDNA is cleaved in only one location. We determine the amount of input eccDNA, transposase used and incubation time to gain an optimal condition. In as experiment, we digested our preparation of eccDNA from HeLa cells, in which approximately 90% of eccDNA were mtDNA, using the NEBNext dsDNA Fragmentase (NEB, M0348S). After 1 minute of digestion with 0.25 ml of the enzyme, we observed a 16 kb fragment on a gel representing mtDNA, demonstrating a single tagmentation.

Sequencing and data analyses. Using the conditions determined by PFGE, we create libraries from human and mouse sperm eccDNA (year 1) and mouse liver (year 2) for the GridION, PromethION, or MinION sequencer, available in the Miller lab at the University of Washington. Either 0.5 ml human semen, 10 million mouse sperm, or 50 mg mouse liver are spiked with control clones. After purification, we run both qPCR- and PFGE-based QCs. Biological duplicates are taken for each sample.

We employ Nanopore ID sequencing chemistry and sequence each strand independently. 1D chemistry routinely achieves very long read lengths from high-quality DNA preparations. Our eccDNA preparation should be high-quality because eccDNA is purified with phenol/chloroform extraction. With the ONT platform and the Rapid Sequencing kit for high-quality human genomic DNA, N50>100 kb and a maximum length of 882 kb were reported. Therefore, we are able to execute full-length sequencing of large eccDNA molecules.

We monitor sequencing runs with MinKNOW. We first validate the successful sequencing of entire lengths of control clones (FIG. 9). We then determine whether each control clone (4.5 kb, 46 kb, and 102 kb) is sequenced for the entire length in a similar efficiency or whether there is a bias toward smaller control clones. Because the same amount (nanogram) is spiked, we expect that, under an unbiased condition, the number of control clones sequenced for the entire length would be inversely correlated with the sizes of clones. We examine the inverse correlation and over/under-representation from the sequencing data (FIG. 9). This analysis is repeated with the various enzyme amounts and digestion durations to optimize the conditions of library construction.

The nanopore ionic current data is base-called using Guppy to generate both FASTQ data and methylation data. The resulting sequences are then aligned to a reference sequence (hg38 and mm10) using Minimap2.

Validation. For endogenous eccDNA, we validate the DNA circularity at the nucleotide level from ONT sequencing data. A read derived from a circular DNA molecule would span a circularizing breakpoint and will be represented as a split read. We determine whether the two alignments assigned are consistent with the circular DNA molecules, as described herein.

DNA methylation One of the advantages of Nanopore sequencing is that base modifications can be called in their native state without enzymatic or chemical treatment prior to sequencing. As a single-DNA molecule travels through a pore, modified bases exhibit unique ionic current signals, which differ from the equivalent unmodified base. Cytosine DNA methylation at CpG dinucleotides (5mC) is a very common base modification and has a strong impact on chromatin compaction and gene transcription.

Among several 5mC callers, Nanopore's Guppy showed superb performance. The superior base-calling model with 5mC is used to base call and extract methylation data for each eccDNA molecule. To validate the calling, we choose 20 eccDNA and use PCR-based amplification of bisulfite modified eccDNA and Sanger-sequencing. Bisulfite modification converts cytosine (C) to uracil, while 5mC remains as C. Uracl will be copied to adenine (A) by PCR whereas C will be copied to Guanine (G), which allows us to distinguish 5mC from C.

### Expected Results and Alternative Approaches

We are developing an unbiased method for eccDNA isolation and a robust method for the characterization of eccDNA in body fluids (human and mouse sperm) and solid tissue (namely mouse liver). By extending our eccDNA isolation protocol of published study for ONT library preparation and sequencing platform, we have shown the characterization: the enrichments of full-length control clones and mtDNA (FIG. 9), single molecule sequencing of large eccDNA for their entire lengths, and CpG methylation calling.

A Column-Free Method for Isolating Large eccDNA Molecules.

Our approach employed an anion-exchange resin column typically used for isolating bacterial plasmids to recover crude eccDNA prior to exonuclease digestion. Anion-exchange columns have been widely used to isolate eccDNA from mammalian tissues/cells. However, the anion-exchange column has a size limit of around 100 kb. In addition to the difficulty in eluting long DNA molecules from the columns, mechanical shearing would be a concern, even though circular DNA is relatively more resistant to mechanical shearing than linear DNA. Indeed, we failed to enrich a 150 kb BAC clone (RP11-615L21) through our column-based procedure.

Because very large eccDNA exists in tissues, particularly tissues with disease conditions such as cancer, an approach suitable for very large eccDNA isolation should be pursued. While not wishing to be bound by any particular theory, we believe that the limitations of the column-based method (difficulty in elution and mechanical shearing) would affect the isolation of very large eccDNA. To test the hypothesis, we develop an anion-exchange column-free approach. We embed cells/nuclei into agarose (FIG. 10). Intact sperm/nuclei immobilized in agarose will be processed to disrupt their cell membrane and remove cellular protein. Subsequently, DNA in the agarose plug will be subject to exonuclease digestion. We have done restriction-enzyme digestion of agarose-embedded cells for 2D agarose gel DNA electrophoresis. With multiple quality control measures (qPCR, PFGE, and Illumina sequencing) for eccDNA enrichments in hand, we rigorously evaluate the purity of eccDNA isolated by column-free methods.

Isolating nuclei is a common approach in experiments with eukaryotes, from yeast, C.elegans, Drosophila to mammals.

Mouse Sperm. We first test the approach for mouse sperm spiked with small and large control clones (>150 kb). Mouse sperm will be swum out into solution from caudae epididymidis and mimics suspension cells, which is suitable for embedding into an agarose plug.

Enrich a clone over 150 kb. First, we directly embed 10 million sperm spiked with control clones into low-melting-point (LMP) agarose. To embed, we suspend sperm in a buffer (50 mM Tris-HCl pH 8.0, 10 mM EDTA, 100 μg/mL RNase A). We mix the sperm with an equal volume of the prewarmed 1% (wt/vol) LMP agarose. Solidified agarose plugs will be transferred into the lysis buffer and incubated at 50° C. for 24 hours. After washing plugs with TE buffer, plugs will be incubated in 1X ExoV buffer and ExoV (NEB) for 24 hours.

We then extract DNA from agarose plugs by digesting agarose with b-agarese I (NEB). After digestion of agarose, we remove the remaining carbohydrate by centrifugation. DNA will be precipitated from supernatant by phenol/chloroform extraction and ethanol precipitation. We will use large pore pipette tips to minimize mechanical shearing.

Mouse liver. Mouse liver serves as a representative solid tissue for evaluating the method for eccDNA extraction. We first isolate nuclei by a published protocol. We homogenize tissues in the homogenization buffer (10 mM Trizma base, 80 mM KCl, 10 mM EDTA, 1 mM spermidine trihydrochloride, 1 mM spermine tetrahydrochloride, 0.5% Triton X-100, pH9.0) and remove intact cells and tissue debris by low-speed centrifugation. From the supernatant, nuclei are pelleted by high-speed centrifugation (2000×g). After counting the nuclei numbers using Trypan blue staining and Countess in our lab, nuclei are embedded into LMP agarose. We then treat the plug with ExoV and recover eccDNA by phenol/chloroform extraction and ethanol precipitation.

It is important to note that the efficient eccDNA extraction from isolated nuclei would open up the method for various organisms regardless of whether or not they have cell walls (for example, yeast and plants) and expand the utility of our method. We could also estimate the abundance of eccDNA in a nucleus, as the number of nuclei going into eccDNA isolation can be counted. We first isolate genomic DNA by the same phenol/chloroform-based extraction from 100,000 nuclei and then estimate the amount of genomic DNA in each nucleus. The total amount of eccDNA/the number of nuclei in a sample/the amount of genomic DNA in each nucleus gives us a direct estimate of the abundance of eccDNA/nuclei.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. An assessment kit for use with a sample containing circular DNA, comprising:
a. a first plasmid DNA molecule which is linearized, or a first plasmid DNA molecule and one or more restriction endonucleases to linearize the first plasmid DNA; and
one or more of:
b. a second plasmid DNA molecule having a size between 1,000 bp and 9,999 bp;
c. a third plasmid DNA molecule having a size between 10,000 bp and 99,999 bp; and
d. a fourth plasmid DNA molecule having a size between 100,000 bp and 300,000 bp.

2. The assessment kit of claim 1, further comprising an exonuclease, wherein the exonuclease is configured to digest linear polynucleotides but does not digest circular DNA, optionally the exonuclease comprising exonuclease V, PLASMID-SAFE™ ATP-Dependent DNase, or both.

3. The assessment kit of claim 1, further comprising a forward primer and a reverse primer for each of the first plasmid DNA molecule and the one or more of the second, the third, and the fourth plasmid DNA molecules: optionally the forward primer and the reverse primer being selected from those disclosed in Table 2.

4. The assessment kit of claim 1, wherein the first plasmid DNA molecule is linearized pEGFP-C1, or wherein the first plasmid DNA molecule is pEGFP-C1 and the restriction endonucleases comprise BamHI and EcoRI; and wherein the second plasmid DNA molecule is pCMV-Cre, the third plasmid DNA molecule is a fosmid, and the fourth plasmid DNA molecule is a bacterial artificial chromosome (BAC) clone.

5. (canceled)

6. (canceled)

7. (canceled)

8. A kit for isolation of extrachromosomal circular DNA (eccDNA) from cells or tissues and assessment of the eccDNA, the kit comprising:
an alkaline buffer for cell lysis;
a proteinase and/or an RNase;
an anion-exchange column or agarose;
a substantially neutral buffer for recovery of covalently closed circular DNA isolated with the anion-exchange column;
an exonuclease, which does not digest circular DNA; and
the assessment kit of claim 1.

9. A method for enriching and quantifying extrachromosomal circular DNA (eccDNA) in a cell or tissue, wherein the cell or tissue comprises chromosomal DNA and the eccDNA, and the eccDNA comprises mitochondrial DNA and non-organelle eccDNA, the method comprising:
(i) lysing the cell or tissue, or obtaining a sample wherein the cell or tissue is lysed; and
(ii) adding to the lysed cell or tissue, or to the sample:
a. a first plasmid DNA molecule which is linearized, and
one or more of:
b. a second plasmid DNA molecule having a size between 1,000 bp and 9,999 bp;
c. a third plasmid DNA molecule having a size between 10,000 bp and 99,999 bp; and
d. a fourth plasmid DNA molecule having a size between 100,000 bp and 300,000 bp;
to obtain a plasmid-spiked sample;
treating the plasmid-spiked sample with a proteinase to degrade protein from the plasmid-spiked sample,
and subsequently treating with an exonuclease to digest linear polynucleotides but not circular DNA, followed by recovery of DNA to obtain the eccDNA; and
(iii) quantifying respective amounts of the first plasmid DNA molecule and the one or more of the second, the third, and the fourth plasmid DNA molecules after the treatment with the exonuclease; and/or quantifying an amount of the mitochondrial DNA and an amount of the chromosomal DNA.

10. The method of claim 9, wherein the method does not include multiple displacement amplification or rolling circle amplification of the cell or tissue, or the sample wherein the cell or tissue is lysed has not been amplified; and wherein the quantification comprises measuring a nucleic acid amount via one or more of quantitative polymerase chain reaction (PCR), next-generation sequencing, and Southern blotting.

11. The method of claim 9, wherein the eccDNA comprises eccDNA of at least 10 kilobases (kb) in size and eccDNA between 0.5 kb and 10 kb; optionally the cell or tissue being a cancerous cell or a tumor tissue.

12. (canceled)

13. The method of claim 9, for enriching and quantifying eccDNA in two or more cells or tissues, wherein a first cell or tissue is a cancerous cell or a tumor tissue, and the second cell or tissue is a normal cell or normal tissue obtained adjacent to the cancerous cell or the tumor tissue.

14. (canceled)

15. The method of claim 9, wherein the quantification further comprises calculating a ratio or relative quantity (RQ) of a target circular DNA over a linear DNA in step (iii);
and the target circular DNA is any one of the one or more of the second, the third, and the fourth plasmid DNA molecule, and the linear DNA is the first plasmid DNA molecule; or the target circular DNA is the mitochondrial DNA, and the linear DNA is a locus of the chromosomal DNA,
optionally the ratio of the target DNA over the linear DNA being greater than 1000, thereby indicating the enrichment of the eccDNA.

16. (canceled)

17. The method of claim 15, wherein the ratio of the target DNA over the linear DNA is greater than 1000, and wherein the linear DNA is chromosome 17 (chr17), and the target circular DNA is chromosome M (chrM).

18. The method of claim 15, wherein the first plasmid DNA molecule is linearized pEGFP-C1, the second plasmid DNA molecule is pCMV-Cre, the third plasmid DNA molecule is a fosmid, and the fourth plasmid DNA molecule is a bacterial artificial chromosome (BAC) clone.

19. (canceled)

20. The method of claim 9, wherein in step (i) lysing the cell or tissue comprises incubating the cell or tissue in a lysis buffer; and step (ii) further comprises treating the plasmid-spiked sample with an RNase, and treating the plasmid-spiked sample with an alkaline buffer following the treatment with the RNase; and wherein the lysis buffer comprises Tris-HCl, EDTA, SDS and NaCl; the treatment with the proteinase comprises adding proteinase K and incubating at about 50° C. for about 2 hours; the treatment with the RNase comprises adding RNase A and incubating at about 50° C. for about 1 hour; and the treatment with the alkaline buffer comprises adding a solution containing NaOH and SDS and incubating for about 5 minutes; and subsequent to step (ii) the method further comprises recovering the eccDNA from the plasmid-spiked sample following the treatment with the exonuclease by using a buffer exchange column, or centrifugal unit.

21. A method for enriching and quantifying extrachromosomal circular DNA (eccDNA) in a cell or tissue, wherein the cell or tissue comprises chromosomal DNA and the eccDNA, and the eccDNA comprises mitochondrial DNA and non-organelle eccDNA, the method comprising:
(i) lysing the cell or tissue, or obtaining a sample wherein the cell or tissue is lysed; and
(ii) adding to the lysed cell or tissue, or to the sample:
a first plasmid DNA molecule which is linearized, and
one or more of:
a second plasmid DNA molecule having a size between 1,000 bp and 9,999 bp;
a third plasmid DNA molecule having a size between 10,000 bp and 99,999 bp; and
a fourth plasmid DNA molecule having a size between 100,000 bp and 300,000 bp,
to obtain a plasmid-spiked sample;
embedding the plasmid-spiked sample into agarose;
digesting the agarose with b-agarese I;
removing any remaining carbohydrate;
precipitating DNA to obtain eccDNA; and
(iii) quantifying respective amounts of the first plasmid DNA molecule and the one or more of the second, the third, and the fourth plasmid DNA molecules; and/or quantifying an amount of the mitochondrial DNA and an amount of the chromosomal DNA.

22. The method of claim 21, further comprising treating the plasmid-spiked sample with a proteinase to degrade protein from the plasmid-spiked sample, and subsequently treating with an exonuclease to digest linear polynucleotides but not circular DNA, before precipitating the DNA.

23. The method of claim 21, wherein the method does not include multiple displacement amplification or rolling circle amplification of the cell or tissue, or the sample wherein the cell or tissue is lysed has not been amplified; and wherein the quantification comprises measuring a nucleic acid amount via one or more of quantitative polymerase chain reaction (PCR), next-generation sequencing, and Southern blotting.

24. The method of claim 21, wherein the eccDNA comprises eccDNA of at least 100 kilobases (kb), or at least 150 kb in size; optionally the cell or tissue being a cancerous cell or a tumor tissue.

25. (canceled)

26. The method of claim 21, for enriching and quantifying eccDNA in two or more cells or tissues, wherein a first cell or tissue is a cancerous cell or a tumor tissue, and the second cell or tissue is a normal cell or normal tissue obtained adjacent to the cancerous cell or the tumor tissue.

27. (canceled)

28. The method of claim 21, wherein the quantification further comprises calculating a ratio or relative quantity (RQ) of a target circular DNA over a linear DNA;
and the target circular DNA is any one of the one or more of the second, the third, and the fourth plasmid DNA molecule, and the linear DNA is the first plasmid DNA molecule; or the target circular DNA is the mitochondrial DNA, and the linear DNA is a locus of the chromosomal DNA,
optionally the ratio of the target DNA over the linear DNA is greater than 1000, thereby indicating the enrichment of the eccDNA.

29. (canceled)

30. The method of claim 28, wherein the ratio of the target DNA over the linear DNA is greater than 1000, and wherein the linear DNA is chromosome 17 (chr17), and the target circular DNA is chromosome M (chrM).

31. The method of claim 28, wherein the first plasmid DNA molecule is linearized pEGFP-C1, the second plasmid DNA molecule is pCMV-Cre, the third plasmid DNA molecule is a fosmid, and the fourth plasmid DNA molecule is a bacterial artificial chromosome (BAC) clone.

32. (canceled)

33. The method of claim 21, wherein in step (i) lysing the cell or tissue comprises incubating the cell or tissue in a lysis buffer; and step (ii) further comprises treating the plasmid-spiked sample with an RNase, and treating the plasmid-spiked sample with an alkaline buffer following the treatment with the RNase; and wherein the lysis buffer comprises Tris-HCl, EDTA, SDS and NaCl; the treatment with the proteinase comprises adding proteinase K and incubating at about 50° C. for about 2 hours; the treatment with the RNase comprises adding RNase A and incubating at about 50° C. for about 1 hour; and the treatment with the alkaline buffer comprises adding a solution containing NaOH and SDS and incubating for about 5 minutes; and subsequent to step (ii) the method further comprises recovering the eccDNA from the plasmid-spiked sample following the treatment with the exonuclease by using a buffer exchange column, centrifugal unit, or an agarose plug.
